(19) United States
(12) Patent Application Publication
(10) Pub. No.: US 2023/0382990 A1
(43) Pub. Date: Nov. 30, 2023

(54) THERAPEUTIC AGENT FOR PERIPARTUM CARDIOMYOPATHY

(71) Applicants: National Cerebral and Cardiovascular Center, Osaka (JP); Chugai Seiyaku Kabushiki Kaisha, Tokyo (JP)

(72) Inventors: Kentaro OTANI, Osaka (JP); Takeshi TOKUDOME, Osaka (JP); Chizuko KAMIYA, Osaka (JP)

(21) Appl. No.: 18/034,429

(22) PCT Filed: Oct. 30, 2020
(86) PCT No.: PCT/JP2020/040927

§ 371 Date: Apr. 28, 2023

(51) Int. Cl. C07K 16/24; A61P 9/04

(52) U.S. Cl. CPC C07K 16/248; A61P 9/04; C07K 2317/24; C07K 2317/21

(57) ABSTRACT

According to the present invention, a pharmaceutical composition for use in the treatment or prevention of peripartum cardiomyopathy, comprising an IL-6 inhibitor as an active ingredient, is provided.

### TECHNICAL FIELD

The present invention relates to a pharmaceutical composition for treating or preventing peripartum cardiomyopathy and methods for treating and preventing peripartum cardiomyopathy.

### BACKGROUND ART

Dilated cardiomyopathy is a group of “idiopathic” cardiomyopathies characterized by (1) myocardial contractile dysfunction and (2) left ventricular dilatation. It is a progressive disease with a poor prognosis characterized by symptoms of chronic heart failure, with repeated acute exacerbations, which may result in sudden death due to fatal arrhythmia, or arterial thromboembolism. There are similar diseases that cause “left ventricular dilatation” and “left ventricular systolic dysfunction” as in dilated cardiomyopathy, and specific cardiomyopathies for which the cause is known are diagnosed as secondary cardiomyopathies and distinguished from idiopathic (primary) dilated cardiomyopathies (Non Patent Literature 1).

Peripartum cardiomyopathy (PPCM, postpartum cardiomyopathy), which is known as a secondary cardiomyopathy, is characterized by the onset of heart failure and the development of a dilated cardiomyopathy-like condition during pregnancy and puerperium in women with no history of heart disease and no other cause for developing heart failure. More than half of patients with peripartum cardiomyopathy will return to normal, but reduced cardiac function will persist in approximately 40%, and severe cases are fatal. Pregnancy and delivery are thought to play a role in the development and progression of the present disease (Non Patent Literature 1). Methods of treatment and pathological studies of peripartum cardiomyopathy have been reported (Non Patent Literatures 2 and 3).

Interleukin 6 (IL-6) is a cytokine also called B-cell stimulating factor 2 (BSF2) or interferon-β2. IL-6 was discovered as a differentiation factor involved in the activation of B lymphocyte lineage cells (Non Patent Literature 4) and was then found to be a multifunctional cytokine that affects various cell functions (Non Patent Literature 5). IL-6 has been reported to induce maturation of T lymphocyte lineage cells (Non Patent Literature 6).

IL-6 transmits its biological activity via two types of proteins on the cell. One is the IL-6 receptor, a ligand-binding protein with a molecular weight of approximately 80 kD, to which IL-6 binds (Non Patent Literatures 7 and 8). The IL-6 receptor exists as a membrane-bound form that penetrates the cell membrane and is expressed on the cell membrane, as well as a soluble IL-6 receptor primarily consisting of the extracellular domain.

The other is gp130, a membrane protein with a molecular weight of approximately 130 kD that is involved in non-ligand-binding signal transduction. IL-6 and the IL-6 receptor form an IL-6/IL-6 receptor complex, which then binds to gp130, thus allowing the biological activity of IL-6 to be transmitted into the cell (Non Patent Literature 9).

Studies have been conducted on the association between IL-6 and various diseases. For example, the association between IL-6 and cardiac hypertrophy and the application of IL-6 inhibitors in the treatment of cardiac diseases have been reported (Non Patent Literatures 10 to 12, and Patent Literatures 1 to 3).

Atrial natriuretic peptide (ANP) is a bioactive peptide consisting of 28 amino acids, that is biosynthesized and stored primarily in the atria and secreted into the blood as needed. ANP has vasodilatory and diuretic properties, and regulates circulatory homeostasis via NPR1 (Natriuretic Peptide Receptor 1), its common receptor with brain natriuretic peptide (BNP). ANP and BNP are already widely applied clinically as diagnostic and therapeutic agents for heart failure.

### CITATION LIST

### Patent Literature

[Patent Literature 1] WO2010/065072 A1
[Patent Literature 2] WO2005/028514 A1
[Patent Literature 3] WO2007/046489 A1

### Non Patent Literature

[Non Patent Literature 1] JCS/JHFS 2018 Guideline on Diagnosis and Treatment of Cardiomyopathies;
[Non Patent Literature 2] Bhattacharyya, A., Tex Heart Inst J. 2012; 39(1): 8-16.
[Non Patent Literature 3] Kurdi, M., Front Immunol. 2018; 9: 3029
[Non Patent Literature 4] Hirano, T. et al., Nature (1986) 324, 73-76
[Non Patent Literature 5] Akira, S. et al., Adv. in Immunology (1993) 54, 1-78
[Non Patent Literature 6] Lotz, M. et al., J. Exp. Med. (1988) 167, 1253-1258
[Non Patent Literature 7] Taga, T. et al., J. Exp. Med. (1987) 166, 967-981
[Non Patent Literature 8] Yamasaki, K. et al., Science (1988) 241, 825-828
[Non Patent Literature 9] Taga, T. et al., Cell (1989) 58, 573-581
[Non Patent Literature 10] Shimizu I., J Mol Cell Cardiol. 2016; 97: 245-262
[Non Patent Literature 11] Chou, C. H. et al., Cardiovascular Research (2018) 114, 690-702
[Non Patent Literature 12] Kang, Y. M. et al., Circ Res. 2006, 99758-766.

### SUMMARY OF INVENTION

### Technical Problem

Both the maternal circulatory system and hormonal balance change dynamically during pregnancy, labor, and post partum. ANP and BNP produced in the heart regulate circulatory homeostasis via NPR1, their common receptor. To clarify the physiological and pathophysiological roles of the endogenous ANP/BNP-NPR1 system in the perinatal period, the phenotype of Npr1-knockout mice during the perinatal period was examined with particular focus on maternal heart weight, blood pressure, and cardiac function. As a result, it was found that Npr1-knockout mice exhibit severe cardiac hypertrophy accompanied by peripartum cardiomyopathy-like fibrosis and that Npr1-knockout mice serve as a model for PPCM without pregnancy-induced hypertension. Hereafter, Npr1-knockout mice may be referred to as Npr1^{-/-} mice and wild-type mice as Npr1^{+/+} mice.

Upon further examination, the present inventors have confirmed that IL-6 mRNA (hereafter, mRNA corresponding to IL-6 may be referred to as 116) expression increases in the hearts of Npr1^{-/-} mice during or after lactation, and found that administration of anti-IL-6 receptor antibodies reduced cardiac hypertrophy in lactating Npr1^{-/-} mice, thereby completing the present invention. According to the present invention, the following compositions, agents, methods, and uses are provided.
[1-1] A pharmaceutical composition for use in the treatment or prevention of peripartum cardiomyopathy, comprising an IL-6 inhibitor as an active ingredient.
[1-2] The pharmaceutical composition according to [1-1], for use in a postpartum or lactating subject.
[1-3] The pharmaceutical composition according to [1-1] or [1-2], wherein the IL-6 inhibitor is an antibody that recognizes IL-6. [1-4] The pharmaceutical composition according to [1-1] or [1-2], wherein the IL-6 inhibitor is an antibody that recognizes an IL-6 receptor.
[1-5] The pharmaceutical composition according to [1-3] or [1-4], wherein the antibody is a monoclonal antibody.
[1-6] The pharmaceutical composition according to any one of [1-3] to [1-5], wherein the antibody is an antibody against human IL-6 or an antibody against a human IL-6 receptor.
[1-7] The pharmaceutical composition according to any one of [1-3] to [1-6], wherein the antibody is a recombinant antibody.
[1-8] The pharmaceutical composition according to any one of [1-3] to [1-7], wherein the antibody is a chimeric antibody, a humanized antibody, or a human antibody.
[1-9] The pharmaceutical composition according to any one of [1-3] to [1-8], wherein the antibody is tocilizumab, satralizumab, or sarilumab.
[2-1] A pharmaceutical composition for use in suppressing or improving cardiac remodeling associated with peripartum cardiomyopathy, comprising an IL-6 inhibitor as an active ingredient.
[2-2] The pharmaceutical composition according to [2-1] for use against cardiac remodeling in a postpartum or lactating subject.
[2-3] The pharmaceutical composition according to [2-1] or [2-2], wherein the IL-6 inhibitor is an antibody that recognizes IL-6.
[2-4] The pharmaceutical composition according to [2-1] or [2-2], wherein the IL-6 inhibitor is an antibody that recognizes an IL-6 receptor.
[2-5] The pharmaceutical composition according to [2-3] or [2-4], wherein the antibody is a monoclonal antibody.
[2-6] The pharmaceutical composition according to any one of [2-3] to [2-5], wherein the antibody is an antibody against human IL-6 or an antibody against a human IL-6 receptor.
[2-7] The pharmaceutical composition according to any one of [2-3] to [2-6], wherein the antibody is a recombinant antibody.
[2-8] The pharmaceutical composition according to any one of [2-3] to [2-7], wherein the antibody is a chimeric antibody, a humanized antibody, or a human antibody.
[2-9] The pharmaceutical composition according to any one of [2-3] to [2-8], wherein the antibody is tocilizumab, satralizumab, or sarilumab.
[2-10] The pharmaceutical composition according to any one of [2-1] to [2-9], for use in the treatment or prevention of peripartum cardiomyopathy.
[3-1] An inhibitor of the IL-6 signaling pathway in the perinatal maternal heart via the neuronal mineralocorticoid receptor, comprising an IL-6 inhibitor as an active ingredient.
[3-2] The inhibitor according to [3-1], for use in a postpartum or lactating subject.
[3-3] The inhibitor according to [3-1] or [3-2], wherein the IL-6 inhibitor is an antibody that recognizes IL-6.
[3-4] The inhibitor according to [3-1] or [3-2], wherein the IL-6 inhibitor is an antibody that recognizes an IL-6 receptor.
[3-5] The inhibitor according to [3-3] or [3-4], wherein the antibody is a monoclonal antibody.
[3-6] The inhibitor according to any one of [3-3] to [3-5], wherein the antibody is an antibody against human IL-6 or an antibody against a human IL-6 receptor.
[3-7] The inhibitor according to any one of [3-3] to [3-6], wherein the antibody is a recombinant antibody.
[3-8] The inhibitor according to any one of [3-3] to [3-7], wherein the antibody is a chimeric antibody, a humanized antibody, or a human antibody.
[3-9] The inhibitor according to any one of [3-3] to [3-8], wherein the antibody is tocilizumab, satralizumab, or sarilumab.
[3-10] The inhibitor according to any one of [3-3] to [3-9], for use in the treatment of peripartum cardiomyopathy postpartum or during lactation.
[4-1] A method for treating or preventing peripartum cardiomyopathy, the method comprising administering an IL-6 inhibitor to a subject in need of the treatment or prevention.
[4-2] The method according to [4-1], for treating or preventing peripartum cardiomyopathy that develops postpartum or during lactation.
[4-3] A method for suppressing or improving cardiac remodeling associated with peripartum cardiomyopathy, the method comprising administering an IL-6 inhibitor to a subject in need of the suppression or improvement.
[4-4] The method according to [4-3], for suppressing or improving cardiac remodeling arising postpartum or during lactation.
[4-5] A method for inhibiting the IL-6 signaling pathway in the perinatal maternal heart via the neuronal mineralocorticoid receptor, the method comprising administering an IL-6 inhibitor to a subject in need of the inhibition.
[4-6] The method according to [4-5], for inhibiting the IL-6 signaling pathway in a postpartum or lactating subject.
[4-7] The method according to any one of [4-1] to [4-6], wherein the IL-6 inhibitor is an antibody that recognizes IL-6.
[4-8] The method according to any one of [4-1] to [4-6], wherein the IL-6 inhibitor is an antibody that recognizes the IL-6 receptor.
[4-9] The method according to [4-7] or [4-8], wherein the antibody is a monoclonal antibody.
[4-10] The method according to any one of [4-7] to [4-9], wherein the antibody is an antibody against human IL-6 or an antibody against a human IL-6 receptor.
[4-11] The method according to any one of [4-7] to [4-10], wherein the antibody is a recombinant antibody.
[4-12] The method according to any one of [4-7] to [4-11], wherein the antibody is a chimeric antibody, a humanized antibody, or a human antibody.
[4-13] The method according to any one of [4-7] to [4-12], wherein the antibody is tocilizumab, satralizumab, or sarilumab.
[5-1] A use of an IL-6 inhibitor in the production of a drug for use in the treatment or prevention of peripartum cardiomyopathy.
[5-2] A use of an IL-6 inhibitor in the production of a drug for use in suppressing or improving cardiac remodeling associated with peripartum cardiomyopathy.
[5-3] A use of an IL-6 inhibitor in the production of a drug for use in suppressing the IL-6 signaling pathway in the perinatal maternal heart via the neuronal mineralocorticoid receptor.
[5-4] The use according to any one of [5-1] to [5-3], wherein the drug is used for a perinatal subject.
[5-5] The use according to any one of [5-1] to [5-4], wherein the IL-6 inhibitor is an antibody that recognizes IL-6.
[5-6] The use according to any one of [5-1] to [5-4], wherein the IL-6 inhibitor is an antibody that recognizes an IL-6 receptor.
[5-7] The use according to any one of [5-1] to [5-6], wherein the antibody is a monoclonal antibody.
[5-8] The use according to any one of [5-1] to [5-7], wherein the antibody is an antibody against human IL-6 or an antibody against a human IL-6 receptor.
[5-9] The use according to any one of [5-1] to [5-8], wherein the antibody is a recombinant antibody.
[5-10] The use according to any one of [5-1] to [5-9], wherein the antibody is a chimeric antibody, a humanized antibody, or a human antibody.
[5-11] The method according to any one of [5-1] to [5-10], wherein the antibody is tocilizumab, satralizumab, or sarilumab.

### Advantageous Effects of Invention

According to the present invention, a therapeutic or prophylactic agent for peripartum cardiomyopathy, particularly a therapeutic or prophylactic agent for peripartum cardiomyopathy postpartum or during lactation is provided.

### BRIEF DESCRIPTION OF DRAWINGS

FIG. 1 is a diagram illustrating the putative mechanism of lactation-induced postpartum cardiomyopathy-like cardiac remodeling in Npr1^{-/-} mice.

FIG. 2A shows an experimental protocol to examine the effect of the number of pregnancy-lactation cycles on the maternal heart. In the present specification, 1PP to 5PP, as shown in the present figure, mean the time of completion of a 4-week lactation period (weaning) in the first to fifth pregnancy-lactation cycles, respectively. Moreover, 1PP mice to 5PP mice mean mice at 1PP to 5PP, respectively.

FIG. 2B is a graph illustrating the effect of repeated pregnancy-lactation cycles on maternal survival. The P value was determined by the log-rank test.

FIG. 2C shows representative photographs of the hearts of Npr1^{+/+} and Npr1^{-/-} mice after 5PP, and a graph of the ratio of heart weight to tibial length (HW/TL) in Npr1^{+/+} and Npr1^{-/-} mice at 5PP. The left graph represents Npr1^{+/+} mice and the right graph represents Npr1^{-/-} mice. Statistical analysis was performed by independent t-test. * indicates P<0.05 vs. 5PP Npr1^{+/+} mice.

FIG. 2D is photographs showing representative examples of the results of staining the hearts of nulliparous (non-pregnant) and 5PP mice by Sirius Red. The scale bar indicates 1 mm.

FIG. 2E is photographs showing representative examples of the results of hematoxylin and eosin staining of hearts in nulliparous mice, 1PP mice and 2PP mice. The scale bar indicates 1 mm.

FIG. 2F is a graph showing the ratio of heart weight to tibial length (HW/TL) in nulliparous mice, 1PP mice, 2PP mice, and 1PP or 2PP mice at 8 weeks postpartum. The left graph represents Npr1^{+/+} mice and the right graph represents Npr1^{-/-} mice. Statistical analysis was performed by two-way analysis of variance with Tukey-Kramer post-test. * indicates P<0.05, †: P<0.05 vs. nulliparous Npr1^{+/+} mice, and ‡: P<0.05 vs. nulliparous Npr1^{-/-} mice.

FIG. 2G is a graph showing the ratio of lung weight to tibial length (LuW/TL) in nulliparous mice, 1PP mice, 2PP mice, and mice at 8 weeks after 1PP or 2PP. The left graph represents Npr1^{+/+} mice and the right graph represents Npr1^{-/-} mice. Statistical analysis was performed by two-way analysis of variance with Tukey-Kramer post-test. * indicates P<0.05, and ‡: P<0.05 vs. nulliparous Npr1^{-/-} mice. NS indicates not significant.

FIG. 2H is photographs showing representative examples of heart histology after Sirius Red staining in nulliparous and 2PP Npr1^{+/+} and Npr1^{-/-} mice. The scale bar indicates 50 μm.

FIG. 2I is a graph showing the quantification of fibrotic regions in the hearts of nulliparous, 1PP and 2PP Npr1^{+/+} and Npr1^{-/-} mice. The left graph represents Npr1^{+/+} mice and the right graph represents Npr1^{-/-} mice. Statistical analysis was performed by two-way analysis of variance with Tukey-Kramer post-test. ‡ indicates P<0.05 vs. nulliparous Npr1^{-/-} mice. NS indicates not significant.

FIG. 2J is photographs showing representative examples of heart histology after staining with fluorescently labeled wheat germ agglutininin in nulliparous and 2PP Npr1^{+/+} and Npr1^{-/-} mice. The scale bar indicates 50 μm.

FIG. 2K is a graph showing the quantification of myocardial cross-sectional area in nulliparous mice, 1PP and 2PP Npr1^{+/+} and Npr1^{-/-} mice. The left graph represents Npr1^{+/+} mice and the right graph represents Npr1^{-/-} mice. Statistical analysis was performed by two-way analysis of variance with Tukey-Kramer post-test. * indicates P<0.05, †: P<0.05 vs. nulliparous Npr1^{-/-} mice, and ‡: P<0.05 vs. nulliparous Npr1^{-/-} mice.

FIG. 3A is a diagram showing an experimental protocol to investigate the effect of lactation on the maternal heart.

FIG. 3B is a graph showing the continuous variation of systolic blood pressure (SBP) in Npr1^{+/+} and Npr1^{-/-} mice during pregnancy. The shading in the graph indicates the gestational period. Statistical analysis was performed by two-way repeated measures analysis of variance. * indicates P<0.05.

FIG. 3C is graphs showing the temporal variation of body weight (BW) and plasma atrial natriuretic peptide (ANP) concentration in Npr1^{+/+} mice (10 to 20 mice per group) and Npr1^{-/-} mice (5 to 13 mice per group) during the gestational and postpartum periods. The shading in the graph indicates the gestational period. Statistical analysis was performed by one-way analysis of variance with Tukey-Kramer post-test. * indicates P<0.05, and t: P<0.05 vs. nulliparous mice.

FIG. 3D is a graph showing the ratio of heart weight to tibial length (HW/TL) in Npr1^{+/+} and Npr1^{-/-} mice when nulliparous, in late pregnancy (E18.5), and immediately after delivery (within 3 days). The left graph represents Npr1^{+/+} mice and the right graph represents Npr1^{-/-} mice. Statistical analysis was performed by two-way analysis of variance with Tukey-Kramer post-test. ‡ indicates P<0.05 vs. corresponding Npr1^{+/+} group. NS indicates not significant.

FIG. 3E is a graph showing the analysis of HW/TL values over time during lactation in Npr1^{+/+} and Npr1^{-/-} mice, and the HW/TL values at 2 weeks postpartum when breastfeeding was avoided (n=8 to 21 in the Npr1^{+/+} group and n=7 to 13 in the Npr1^{-/-} group). Statistical analysis was performed by two-way analysis of variance with Tukey-Kramer post-test. † indicates P<0.05 vs. nulliparous mice, and ‡: P<0.05 vs. corresponding Npr1^{+/+} group.

FIG. 3F is a graph showing the relative expression levels of genes involved in cardiac hypertrophy and cardiac fibrosis in the hearts of 2-week lactating Npr1^{+/+} and Npr1^{-/-} mice (n=8 mice per group). From left to right, respectively, the graphs of nulliparous Npr1^{+/+} mice, nulliparous Npr1^{-/-} mice, 2-week lactating Npr1^{+/+} mice, and 2-week lactating Npr1^{-/-} mice are represented. Statistical analysis was performed by two-way analysis of variance with Tukey-Kramer post-test. * indicates P<0.05.

FIG. 4A shows an experimental protocol to examine the involvement of aldosterone, IL-6, sympathetic or parasympathetic activity, and oxidative stress in lactation-induced cardiac hypertrophy of Npr1^{-/-} mice. The present experiment showed that IL-6-induced inflammation is involved in lactation-induced cardiac hypertrophy in Npr1^{-/-} mice.

FIG. 4B is a graph showing the relative gene expression levels of IL-6 and IL-1β in the hearts of nulliparous (non-pregnant) or 2-week lactating Npr1^{+/+} and Npr1^{-/-} mice. Statistical analysis was performed by two-way analysis of variance with Tukey-Kramer post-test. * indicates P<0.05. NS indicates not significant.

FIG. 4C is a diagram showing the effect of lactation on the phosphorylation of signal-transducing transcription factor 3 (STAT3) protein in the hearts of Npr1^{+/+} and Npr1^{-/-} mice.

FIG. 4D is a graph showing the relative gene expression levels of IL-6 and IL-1β in the hearts of 2-week lactating Npr1^{+/+} and Npr1^{-/-} mice given a control diet or a diet containing eplerenone, a mineralocorticoid receptor (MR) antagonist. Statistical analysis was performed by two-way analysis of variance with Tukey-Kramer post-test. NS indicates not significant.

FIG. 4E is a graph showing the ratio of heart weight to tibial length (HW/TL) in 2-week lactating Npr1^{+/+} and Npr1^{-/-} mice with administration of control immunoglobulin G (IgG) or MR16-1 (anti-IL-6 receptor antibody). Statistical analysis was performed by two-way analysis of variance with Tukey-Kramer post-test. NS indicates not significant.

FIG. 4F is a graph showing a comparison of HW/TL in 2-week lactating Npr1^{-/-} mice with and without administration of metoprolol (β1 receptor antagonist), nicotine (parasympathomimetic drug), or tempol (radical scavenger). Statistical analysis was performed by one-way analysis of variance with Tukey-Kramer post-test. NS indicates not significant.

FIG. 4G is a graph showing a comparison of HW/TL in nulliparous (open square) and 2-week lactating (solid square) mice with tissue-specific deletion of Npr1 (nestin, neurons; αMHC, cardiomyocytes; Tie2, endothelial cells; AQP2, collecting ducts; and CLC-KB, distal tubules) and Npr1^{-/-} mice. Statistical analysis was performed by one-way analysis of variance with Dunnett post-test and independent t-test. * indicates P<0.05. NS indicates not significant.

### DESCRIPTION OF EMBODIMENTS

The present invention relates to a therapeutic or prophylactic agent for peripartum cardiomyopathy, comprising an IL-6 inhibitor as an active ingredient.

In the present invention, the “IL-6 inhibitor” is a substance that blocks signal transduction by IL-6 and inhibits the biological activity of IL-6. The IL-6 inhibitor is preferably a substance having an inhibitory effect on the binding of either IL-6, an IL-6 receptor, or gp130.

Examples of the IL-6 inhibitor of the present invention include, but are not limited to, anti-IL-6 antibodies, anti-IL-6 receptor antibodies, anti-gp130 antibodies, IL-6 variants, soluble IL-6 receptor variants, partial peptides of IL-6 and IL-6 receptor, and low molecular weight substances exhibiting activities similar thereto. Preferred examples of the IL-6 inhibitor of the present invention include an antibody that recognizes an IL-6 receptor.

The origin of the antibody in the present invention is not particularly limited, but preferred examples include antibodies of mammalian origin, and more preferably of human origin.

The anti-IL-6 antibody used in the present invention can be obtained as a polyclonal or a monoclonal antibody by use of an approach known in the art. As the anti-IL-6 antibody used in the present invention, a mammal-derived monoclonal antibody is particularly preferable. The mammal-derived monoclonal antibody includes those produced by hybridomas and those produced by hosts transformed with expression vectors containing an antibody gene by a genetic engineering approach. This antibody binds to IL-6, thereby inhibiting the binding of IL-6 to the IL-6 receptor and thus blocking the transmission of IL-6's biological activity into the cell.

Examples of such antibody include MH166 (Matsuda, T. et al., Eur. J. Immunol. (1988) 18, 951-956) and SK2 antibody (Sato, K. et al., The abstracts of the 21st Annual Meeting of the Japanese Society for Immunology (1991) 21, 166).

Anti-IL-6 antibody-producing hybridomas can basically be prepared as follows by use of a technique known in the art. That is, they can be prepared by using IL-6 as a sensitizing antigen to immunize them according to a usual immunization method, fusing the obtained immunocytes with parental cells known in the art by a usual cell fusion method, and screening the monoclonal antibody-producing cells by a usual screening method.

Specifically, anti-IL-6 antibodies can be prepared as follows. For example, human IL-6 for use as a sensitizing antigen for antibody obtainment is obtained by using the IL-6 gene/amino acid sequence disclosed in Eur. J. Biochem (1987) 168, 543-550, J. Immunol. (1988) 140, 1534-1541, or Agr. Biol. Chem. (1990) 54, 2685-2688.

After inserting the IL-6 gene sequence into an expression vector system known in the art and transforming appropriate host cells, the IL-6 protein of interest can be purified from the host cells or culture supernatant by a method known in the art, and this purified IL-6 protein can be used as a sensitizing antigen. Fusion proteins of an IL-6 protein with other proteins may also be used as sensitizing antigens.

The anti-IL-6 receptor antibody used in the present invention can be obtained as a polyclonal or a monoclonal antibody by use of an approach known in the art. As the anti-IL-6 receptor antibody used in the present invention, a mammal-derived monoclonal antibody is particularly preferable. The mammal-derived monoclonal antibody includes those produced by hybridomas and those produced by hosts transformed with expression vectors containing an antibody gene by a genetic engineering approach. This antibody binds to the IL-6 receptor, thereby inhibiting the binding of IL-6 to the IL-6 receptor and thus blocking the transmission of IL-6's biological activity into the cell.

Examples of such antibody include the MR16-1 antibody (Tamura, T. et al. Proc. Natl. Acad. Sci. USA (1993) 90, 11924-11928), PM-1 antibody (Hirata, Y. et al., J. Immunol. (1989) 143, 2900-2906), AUK12-20 antibody, AUK64-7 antibody, and AUK146-15 antibody (International Patent Application Publication No. WO 92-19759). Among these, preferred examples of the monoclonal antibody against the human IL-6 receptor include the PM-1 antibody, and preferred examples of the monoclonal antibody against the mouse IL-6 receptor include the MR16-1 antibody.

Anti-IL-6 receptor monoclonal antibody-producing hybridomas can basically be prepared as follows by use of a technique known in the art. That is, they can be prepared by using the IL-6 receptor as a sensitizing antigen to immunize them according to a usual immunization method, fusing the obtained immunocytes with parental cells known in the art by a usual cell fusion method, and screening the cells producing a monoclonal antibody by a usual screening method.

Specifically, anti-IL-6 receptor antibodies can be prepared as follows. For example, the human IL-6 receptor for use as a sensitizing antigen for antibody obtainment is obtained by using the IL-6 receptor gene/amino acid sequence disclosed in European Patent Application Publication No. EP 325474, and for the mouse IL-6 receptor, the IL-6 receptor gene/amino acid sequence disclosed in Japanese Patent Application Publication No. 3-155795.

There are two types of IL-6 receptor proteins: those expressed on the cell membrane and those that are detached from the cell membrane (soluble IL-6 receptors) (Yasukawa, K. et al., J. Biochem. (1990) 108, 673-676). The soluble IL-6 receptor differs from the membrane-bound IL-6 receptor in that it consists substantially of the extracellular domain of the IL-6 receptor bound to the cell membrane and lacks the transmembrane domain or the transmembrane domain and intracellular domain. Any IL-6 receptor protein may be used as long as it can be used as a sensitizing antigen for the preparation of anti-IL-6 receptor antibodies for use in the present invention.

After inserting the IL-6 receptor gene sequence into an expression vector system known in the art and transforming appropriate host cells, the IL-6 receptor protein of interest can be purified from the host cells or culture supernatant by a method known in the art, and this purified IL-6 receptor protein can be used as a sensitizing antigen. Cells expressing the IL-6 receptor and fusion proteins of an IL-6 receptor protein with other proteins may also be used as sensitizing antigens.

The anti-gp130 antibody used in the present invention can be obtained as a polyclonal or a monoclonal antibody by use of an approach known in the art. As the anti-gp130 antibody used in the present invention, a mammal-derived monoclonal antibody is particularly preferable. The mammal-derived monoclonal antibody includes those produced by hybridomas and those produced by hosts transformed with expression vectors containing an antibody gene by a genetic engineering approach. This antibody binds to gp130, thereby inhibiting the binding of the IL-6/IL-6 receptor complex to gp130 and thus blocking the transmission of IL-6's biological activity into the cell.

Examples of such antibody include the AM64 antibody (Japanese Patent Laid-Open No. 3-219894), 4B11 and 2H4 antibodies (U.S. Pat. No. 5,571,513), and B-S12 and B-P8 antibodies (Japanese Patent Laid-Open No. 8-291199).

Anti-gp130 monoclonal antibody-producing hybridomas can basically be prepared as follows by use of a technique known in the art. That is, they can be prepared by using gp130 as a sensitizing antigen to immunize them according to a usual immunization method, fusing the obtained immunocytes with parental cells known in the art by a usual cell fusion method, and screening the monoclonal antibody-producing cells by a usual screening method.

Specifically, monoclonal antibodies can be prepared as follows. For example, gp130 for use as a sensitizing antigen for antibody obtainment is obtained by using the gp130 gene/amino acid sequence disclosed in European Patent Application Publication No. EP 411946.

After inserting the gp130 gene sequence into an expression vector system known in the art and transforming appropriate host cells, the gp130 protein of interest can be purified from the host cells or culture supernatant by a method known in the art, and this purified gp130 protein can be used as a sensitizing antigen. Cells expressing gp130 and fusion proteins of an gp130 protein with other proteins may also be used as sensitizing antigens.

The mammals to be immunized with the sensitizing antigen are not particularly limited, and are preferably selected in consideration of compatibility with the parental cells for use in cell fusion. In general, rodents, such as mice, rats, and hamsters, are used.

Immunizing animals with sensitizing antigens is done according to a method known in the art. For example, a general method is to inject the sensitizing antigen intraperitoneally or subcutaneously into the mammal. Specifically, it is preferable to dilute the sensitizing antigen with PBS (phosphate-buffered saline), saline, or the like to an appropriate amount, mix the suspension with an appropriate amount of a usual adjuvant, for example, a Freund's complete adjuvant, if desired, emulsify it, and then administer it to the mammals several times at 4- to 21-day intervals. Appropriate carriers can also be used when immunizing with a sensitizing antigen.

After such immunization and confirmation of elevated levels of the desired antibodies in the serum, the immunocytes are removed from the mammal and subjected to cell fusion. Preferred examples of immunocytes to be subjected to cell fusion particularly include spleen cells.

For the mammalian myeloma cells as the other parental cell to be fused with the immunocytes, various cell lines known in the art, such as P3X63Ag8.653 (Kearney, J. F. et al. J. Immunol. (1979) 123, 1548-1550), P3X63Ag8U.1 (Current Topics in Microbiology and Immunology (1978) 81, 1-7), NS-1 (Kohler. G. and Milstein, C. Eur. J. Immunol. (1976) 6, 511-519), MPC-11 (Margulies D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21), 5194 (Trowbridge, I. S., J. Exp. Med. (1978) 148, 313-323), and R210 (Galfre, G. et al., Nature (1979) 277, 131-133), are already appropriately used.

Basically, the cell fusion of the immunocytes with the myeloma cells can be carried out according to a method known in the art, for example, the method of Milstein et al. (Kohler. G. and Milstein, C., Methods Enzymol. (1981) 73, 3-46).

More specifically, the cell fusion is carried out, for example, in a usual nutrient medium in the presence of a cell fusion promoter. For example, polyethylene glycol (PEG) or hemagglutinating virus of Japan (HVJ) is used as the fusion promoter. In addition, an auxiliary such as dimethyl sulfoxide can also be added, if desired, for enhancing fusion efficiency.

The ratio of immunocytes to myeloma cells used is preferably set to, for example, 1 to 10-fold. For example, an RPMI1640 medium or a MEM medium suitable for the growth of the myeloma cell line as well as a usual medium for use in this kind of cell culture can be used as the medium for use in the cell fusion, and furthermore, a solution supplemented with serum such as fetal calf serum (FCS) can also be used in combination.

In cell fusion, the fusion cells (hybridomas) of interest are formed by mixing well predetermined amounts of the immunocytes and myeloma cells in the medium, adding a PEG solution, for example, a PEG solution with an average molecular weight of the order of 1000 to 6000, preheated to approximately 37° C. usually at a concentration of 30 to 60% (w/v), and mixing. Subsequently, an appropriate medium is sequentially added, and its supernatant is removed by centrifugation. This operation can be repeated to remove the cell fusion agents or the like unfavorable for hybridoma growth.

The hybridomas can be cultured in a usual selective medium, for example, a HAT medium (medium containing hypoxanthine, aminopterin, and thymidine), for selection. The culture in the HAT medium is continued for a time long enough to kill cells (non-fused cells) other than the hybridomas of interest, usually several days to several weeks. Subsequently, hybridomas producing the antibody of interest are screened for and cloned by performing a usual limiting dilution method.

In addition to immunizing non-human animals with an antigen to obtain the above hybridomas, human lymphocytes can also be sensitized with the desired antigen protein or antigen-expressing cells in vitro, and the sensitized B lymphocytes can be fused with human myeloma cells, such as U266, to obtain the desired human antibody having binding activity to the desired antigen or antigen-expressing cells (see Japanese Patent Publication No. 1-59878). Furthermore, an antigen or antigen-expressing cells may be administered to transgenic animals having a repertoire of human antibody genes to obtain the desired human antibody according to the method described above (see International Patent Application Publication Nos. WO 93/12227, WO 92/03918, WO 94/02602, WO 94/25585, WO 96/34096, and WO 96/33735).

The monoclonal antibody-producing hybridomas thus prepared can be subcultured in a usual medium and can also be stored over a long period in liquid nitrogen.

To obtain the monoclonal antibody from the hybridomas, methods such as culturing the hybridomas according to a usual method and obtaining the antibody as the culture supernatant, or growing the hybridomas by administering them to mammals compatible therewith and obtaining the antibody as the ascitic fluids thereof, are adopted. The former method is suitable for obtaining highly pure antibodies, while the latter method is suitable for mass production of antibodies.

For example, anti-IL-6 receptor antibody-producing hybridomas can be prepared by the methods disclosed in Japanese Patent Laid-Open No. 3-139293. They can be prepared by a method of injecting PM-1 antibody-producing hybridomas intraperitoneally into BALB/c mice, obtaining ascites fluid, and purifying the PM-1 antibodies from this ascites fluid, or a method of culturing the present hybridomas in an appropriate medium, such as RPMI 1640 medium containing 10% fetal bovine serum and 5% BM-Condimed H1 (manufactured by Boehringer Mannheim), Hybridoma-SFM medium (manufactured by GIBCO-BRL), or PFHM-II medium (manufactured by GIBCO-BRL), and purifying the PM-1 antibodies from the culture supernatant.

In the present invention, recombinant antibodies produced by cloning antibody genes from hybridomas, incorporating them into appropriate vectors, introducing them into hosts, and using a genetic recombination technique, can be used as monoclonal antibodies (see, for example, Borrebaeck C. A. K. and Larrick J. W. THERAPEUTIC MONOCLONAL ANTIBODIES, Published in the United Kingdom by MACMILLAN PUBLISHERS LTD, 1990).

Specifically, mRNA encoding a variable (V) region of the antibody of interest is isolated from cells producing the antibody, e.g., hybridomas. For the isolation of mRNA, total RNA is prepared by a method known in the art, such as the guanidine ultracentrifugation method (Chirgwin, J. M. et al., Biochemistry (1979) 18, 5294-5299) or AGPC method (Chomczynski, P. et al., Anal. Biochem. (1987) 162, 156-159), and mRNA is prepared using an mRNA Purification Kit (manufactured by Pharmacia) or the like. mRNA can also be prepared directly by using QuickPrep mRNA Purification Kit (manufactured by Pharmacia).

From the obtained mRNA, the cDNA for the antibody V region is synthesized using reverse transcriptase. The cDNA can be synthesized using AMV Reverse Transcriptase First-strand cDNA Synthesis Kit and the like. In addition, the 5′-Ampli FINDER RACE Kit (manufactured by Clontech) and the 5′-RACE method using PCR (Frohman, M. A. et al., Proc. Natl. Acad. Sci. USA (1988) 85, 8998-9002; Belyaysky, A. et al., Nucleic Acids Res. (1989) 17, 2919-2932) can be used to synthesize and amplify cDNA. The DNA fragment of interest is purified from the obtained PCR products and ligated with a vector DNA. Furthermore, recombinant vectors are then created, introduced into E. coli or the like and colonies are selected to prepare the desired recombinant vector. The nucleotide sequence of the DNA of interest is confirmed by a method known in the art, e.g., the deoxy method.

Once the DNA encoding the V region of the antibody of interest is obtained, it is linked to the DNA encoding the antibody constant region (C region) of interest and incorporated into an expression vector. Alternatively, DNA encoding the V region of the antibody may be incorporated into an expression vector containing DNA for the antibody C region.

To produce the antibody used in the present invention, the antibody gene is incorporated into an expression vector so that it is expressed under the control of expression control regions, e.g., enhancers and promoters, as described below. Host cells can then be transformed with this expression vector to express the antibody.

In the present invention, genetically recombinant antibodies that have been altered artificially for the purpose of reducing heteroantigenicity in humans, for example, chimeric antibodies, humanized antibodies, and human antibodies, can be used. Such altered antibodies can be produced using a known method.

Chimeric antibodies are obtained by linking DNA encoding the antibody V region obtained as described above with DNA encoding a human antibody C region, which is then incorporated into an expression vector, and introduced into a host for production (see European Patent Application Publication No. EP 125023 and International Patent Application Publication No. WO 92-19759). This known method can be used to obtain chimeric antibodies useful for the present invention.

Humanized antibodies, also referred to as reshaped human antibodies or humanized antibodies, are non-human mammalian, e.g., mice antibodies in which the complementarity determining region (CDR) is grafted to the complementarity determining region of a human antibody, and the general gene recombination approaches are also known (see European Patent Application Publication No. EP 125023 and International Patent Application Publication No. WO 92-19759).

Specifically, a DNA sequence designed to link the CDR of a mouse antibody to the framework region (FR) of a human antibody is synthesized by PCR from several oligonucleotides prepared so as to have overlapping portions at the ends. The antibodies are obtained by linking the obtained DNA with DNA encoding a human antibody C region, which is then incorporated into an expression vector, and introduced into a host for production (see European Patent Application Publication No. EP 239400 and International Patent Application Publication No. WO 92-19759).

As the FR of a human antibody to be linked via CDR, an FR whose complementarity determining region form a good antigen binding site is selected. If necessary, amino acids in the framework region of the variable region of the antibody may be replaced so that the complementarity determining region of the reshaped human antibody forms an appropriate antigen binding site (Sato, K. et al., Cancer Res. (1993) 53, 851-856).

Human antibody C regions are used for chimeric and humanized antibodies. Human antibody C regions include Cy, and, for example, C71, C72, C73 or C74 can be used. A human antibody C region may also be modified to improve the stability of the antibody or its production.

A chimeric antibody consists of variable regions of an antibody derived from a non-human mammal and C regions derived from a human antibody. A humanized antibody consists of complementarity determining regions of an antibody derived from a non-human mammal and framework regions and C regions derived from a human antibody. These have reduced antigenicity in the human body and are therefore useful as the antibody used in the present invention.

Preferred specific examples of the humanized antibody used in the present invention includes humanized PM-1 antibody (see International Patent Application Publication No. WO 92-19759).

As a method for obtaining a human antibody, a technique of obtaining a human antibody by panning using a human antibody library is also known in addition to the methods mentioned above. For example, human antibody variable regions can be expressed as a single-chain antibody (scFv) on the surface of phages by a phage display method, and the phage binding to the antigen can be selected. The gene of the selected phage can be analyzed to determine DNA sequences encoding the variable regions of the human antibody binding to the antigen. Once the DNA sequence of the scFv binding to the antigen is identified, an appropriate expression vector containing the sequence can be prepared to obtain the human antibody. These methods are already well known, and WO 92/01047, WO 92/20791, WO 93/06213, WO 93/11236, WO 93/19172, WO 95/01438, and WO 95/15388 can be referred to.

An antibody gene constructed as described above can be expressed by a method known in the art. When mammalian cells are used, the gene can be expressed by DNA to which are functionally bound a commonly used useful promoter, the antibody gene to be expressed, and a polyA signal downstream of the 3′ side, or a vector containing the same. Examples of the promoter/enhancer include the human cytomegalovirus immediate early promoter/enhancer. As other promoters/enhancers that can be used to express the antibody used in the present invention, viral promoters/enhancers of retrovirus, polyomavirus, adenovirus, simian virus 40 (SV40), and the like, and promoters/enhancers derived from mammalian cells such as human elongation factor 1α (HEF1α) can be used.

Antibody expression can be easily carried out by following, for example, the method of Mulligan et al. (Mulligan, R. C. et al., Nature (1979) 277, 108-114), if using the SV40 promoter/enhancer, and the method of Mizushima et al. (Mizushima, S. and Nagata, S. Nucleic Acids Res. (1990) 18, 5322), if using the HEF1α promoter/enhancer.

In the case of E. coli, the antibody can be expressed by functionally binding a commonly used useful promoter, a signal sequence for antibody secretion, and the antibody gene to be expressed. Example of the promoter include the lacZ promoter and the araB promoter. If using the lacZ promoter, the method of Ward et al. (Ward, E. S. et al., Nature (1989) 341, 544-546; Ward, E. S. et al. FASEB J. (1992) 6, 2422-2427) can be followed, and if using the araB promoter, the method of Better et al. (Better, M. et al. Science (1988) 240, 1041-1043) can be followed.

As a signal sequence for antibody secretion, the pelB signal sequence (Lei, S. P. et al J. Bacteriol. (1987) 169, 4379-4383) can be used if produced in the periplasm of E. coli. After isolating the antibodies produced in the periplasm, the antibody structure is appropriately refolded and used (see, for example, WO96/30394).

As the replication origin, those derived from SV40, poliomavirus, adenovirus, bovine papillomavirus (BPV), and the like can be used. Furthermore, for gene copy number amplification in the host cell system, the expression vector can contain the aminoglycoside phosphotransferase (APH) gene, thymidine kinase (TK) gene, E. coli xanthine-guanine phosphoribosyltransferase (Ecogpt) gene, dihydrofolate reductase (dhfr) gene, and the like as a selection marker.

Any production system can be used for the production of the antibody used in the present invention. The production systems for antibody production include in vitro and in vivo production systems. Examples of in vitro production systems include production systems using eukaryotic cells and production systems using prokaryotic cells.

When eukaryotic cells are used, these include production systems using animal cells, plant cells, or fungus cells. As animal cells, (1) mammalian cells, such as CHO, COS, myeloma, BHK (baby hamster kidney), HeLa, and Vero, (2) amphibian cells, such as Xenopus oocytes, or (3) insect cells, such as sf9, sf21, and Tn5 are known. As plant cells, cells derived from Nicotiana tabacum are known, which can be cultured in callus culture. As fungus cells, yeast, for example, of the genus Saccharomyces, such as Saccharomyces cerevisiae, filamentous fungi, for example, of the genus Aspergillus, such as Aspergillus niger, and the like are known.

When prokaryotic cells are used, the production systems include production systems using bacterial cells. E. coli and Bacillus subtilis are known as bacterial cells.

Antibodies are obtained by introducing the antibody gene of interest into these cells by transformation and culturing the transformed cells in vitro. Culture is performed according to a method known in the art. For example, DMEM, MEM, RPMI 1640, and IMDM can be used as the culture solution, and a solution supplemented with serum such as fetal calf serum (FCS) can also be used in combination. Antibodies may also be produced in vivo by transferring cells into which the antibody gene has been introduced into the abdominal cavity or the like of an animal.

On the other hand, examples of in vivo production systems include production systems using animals and production systems using plants. When animals are used, these include production systems using mammals and insects.

A goat, pig, sheep, mouse, cow or the like can be used as the mammal (Vicki Glaser, SPECTRUM Biotechnology Applications, 1993). As insects, silkworms can be used. If plants are used, for example, tobacco can be used.

Antibody genes are introduced into these animals or plants to produce antibodies inside the animal's or plant's body, which are then collected. For example, the antibody gene is inserted in the middle of a gene encoding a protein specifically produced in milk, such as goat β casein, to thereby prepare a fusion gene. A DNA fragment containing the fusion gene having the antibody gene insert is injected into goat embryos, which are in turn introduced into female goats. The desired antibody is obtained from the milk produced by transgenic goats brought forth by the goats that have received the embryo, or progeny thereof. In order to increase the amount of milk containing the desired antibody produced from the transgenic goats, hormone can be appropriately used on the transgenic goats (Ebert, K. M. et al., Bio/Technology (1994) 12, 699-702).

When using silkworms, the silkworms are infected with a baculovirus in which the antibody gene of interest has been inserted, and the desired antibody is obtained from the body fluid of the silkworms (Maeda, S. et al., Nature (1985) 315, 592-594). Furthermore, when using tobacco, the antibody gene of interest is inserted into a vector for plant expression, e.g., pMON530, and this vector is introduced into bacteria such as Agrobacterium tumefaciens. Tobacco, e.g., Nicotiana tabacum, is infected with these bacteria, and the desired antibody is obtained from the leaves of the present tobacco (Julian, K.-C. Ma et al., Eur. J. Immunol. (1994) 24, 131-138).

When producing antibodies in an in vitro or in vivo production system as described above, DNA encoding an antibody heavy chain (H chain) or light chain (L chain) may be incorporated into separate expression vectors and the host transformed simultaneously, or DNA encoding the H and L chains may be incorporated into a single expression vector and the host transformed (see International Patent Application Publication No. WO 94-11523).

The antibody used in the present invention may be a full-length antibody, a fragment of antibody, or a modified product thereof, as long as it can be suitably used in the present invention. A “full-length antibody” indicates an antibody consisting of two “full-length antibody heavy chains” and two “full-length antibody light chains”. A “full-length antibody heavy chain” is a polypeptide consisting of an antibody heavy chain variable domain (VH), antibody heavy chain constant domain 1 (CH1), antibody hinge region (HR), antibody heavy chain constant domain 2 (CH2), and antibody heavy chain constant domain 3 (CH3) (abbreviated as VH-CH1-HR-CH2-CH3) from the N terminus to the C terminus. A “full-length antibody light chain” is a polypeptide consisting of an antibody light chain variable domain (VL) and an antibody light chain constant domain (CL) (abbreviated as VL-CL) from the N terminus to the C terminus. The antibody light chain constant domain (CL) can be κ (kappa) or λ (lambda). Two full-length antibody chains are linked together between the CL and CH1 domains and between the hinge regions in the full-length antibody heavy chains via inter-polypeptide disulfide bonds. Examples of typical full-length antibodies are natural antibodies such as IgG (e.g., IgG1 and IgG2), IgM, IgA, IgD, and IgE. Examples of fragments of antibodies include Fab, F(ab′)₂, Fv, and single chain Fv (scFv) in which H chain and L chain Fvs are linked by an appropriate linker.

Specifically, an antibody is treated with an enzyme such as papain and pepsin to generate antibody fragments, or genes encoding such antibody fragments are constructed, these are introduced into an expression vector, and then expressed in an appropriate host cell (e.g., see Co, M. S. et al., J. Immunol. (1994) 152, 2968-2976; Better, M. & Horwitz, A. H. Methods in Enzymology (1989) 178, 476-496; Plueckthun, A. & Skerra, A. Methods in Enzymology (1989) 178, 497-515; Lamoyi, E., Methods in Enzymology (1989) 121, 652-663, Rousseaux, J. et al., Methods in Enzymology (1989) 121, 663-66; Bird, R. E. et al., TIBTECH (1991) 9, 132-137).

scFv is obtained by linking the H chain V region and L chain V region of an antibody. In this scFv, the H chain V region and L chain V region are linked via a linker, preferably via a peptide linker (Huston, J. S. et al. Proc. Natl. Acad. Sci. U.S.A. (1988) 85, 5879-5883). The H chain V region and L chain V region in scFv may be derived from any of the antibodies described above. As the peptide linker linking the V region, for example, any single chain peptide consisting of 12 to 19 amino acid residues is used.

DNA encoding scFv is obtained by using DNA encoding the H chain or H chain V region and DNA encoding the L chain or L chain V region of the antibody as templates, amplifying the DNA portion encoding the desired amino acid sequence among those sequences by PCR using primer pairs defining both ends thereof, and then further amplifying by combining the DNA encoding the peptide linker portion and primer pairs that define both ends thereof to be linked to the H and L chains, respectively.

Once DNAs encoding scFv are prepared, expression vectors containing the DNAs and hosts transformed by these expression vectors can be obtained according to a conventional method, and scFv can be obtained using the hosts according to a conventional method.

These antibody fragments can be produced by a host by obtaining and expressing their genes in the same manner as described above. “Antibody” as used in the present invention also includes these antibody fragments.

Antibodies bound to various molecules such as polyethylene glycol (PEG) can also be used as a modified product of an antibody. “Antibody” as used in the present invention also includes these modified products of antibodies. Such modified product of an antibody can be obtained by chemically modifying the obtained antibody. These methods have already been established in the art.

Antibodies produced and expressed as described above can be purified to homogeneity by separation from the host, both inside and outside the cell. Separation and purification of the antibody used in the present invention can be performed by affinity chromatography. Examples of the column used in affinity chromatography include a protein A column and a protein G column. Examples of the carrier used in a protein A column include HyperD, POROS, and Sepharose F.F. Beyond this, the usual separation and purification methods used for proteins can be used, and are not limited in any way.

For example, chromatography other than the affinity chromatography described above, filters, ultrafiltration, salting-out, dialysis, and the like can be appropriately selected and combined to separate and purify the antibody used in the present invention. Examples of the chromatography includes ion exchange chromatography, hydrophobic chromatography, and gel filtration. These chromatographies can be applied to HPLC (high performance liquid chromatography). In addition, reverse phase HPLC may also be used.

The concentration of the antibody obtained above can be measured by absorbance measurement, ELISA, or the like. That is, if using absorbance measurement, after appropriate dilution with PBS(−), the absorbance at 280 nm is measured and the concentration is calculated with 1 mg/ml as 1.35 OD. If using ELISA, the measurement can be done as follows. That is, 100 μl of goat anti-human IgG (manufactured by TAG) diluted to 1 vg/ml with 0.1 M bicarbonate buffer (pH 9.6) is added to a 96-well plate (manufactured by Nunc) and incubated at 4° C. overnight to solidify the antibody. After blocking, 100 μl of the antibody used in the present invention, a sample containing the antibody, or human IgG (manufactured by CAPPEL) as a standard product, diluted as appropriate, is added and incubated at room temperature for 1 hour.

After washing, 100 μl of alkaline phosphatase-labeled anti-human IgG (manufactured by BIOSOURCE) diluted 5000-fold is added and incubated at room temperature for 1 hour. After washing, a substrate solution is added, and following incubation, the absorbance at 405 nm is measured using a MICROPLATE READER Model 3550 (manufactured by Bio-Rad) to calculate the concentration of the antibody of interest.

The IL-6 variant used in the present invention is a substance that has binding activity with the IL-6 receptor and does not transmit the biological activity of IL-6. That is, the IL-6 variant binds to the IL-6 receptor competitively with IL-6, but does not transmit the biological activity of IL-6, and thus blocks signal transduction by IL-6.

The IL-6 variant is prepared by introducing mutations by replacing amino acid residues in the amino acid sequence of IL-6. The IL-6 from which the IL-6 variant is made can be of any origin, but is preferably human IL-6 in consideration of antigenicity and the like.

Specifically, this is performed by predicting the secondary structure of the amino acid sequence of IL-6 using a molecular modeling program known in the art, e.g., WHATIF (Vriend et al., J. Mol. Graphics (1990) 8, 52-56) and then evaluating the effect of the substituted amino acid residue on the whole. After determining the appropriate substituted amino acid residue, a vector containing a nucleotide sequence encoding the human IL-6 gene is used as a template to introduce the mutation so that the amino acid is substituted by a PCR method usually performed, and thereby obtain a gene encoding the IL-6 variant. This gene can be incorporated into an appropriate expression vector if necessary to obtain the IL-6 variant in accordance with the methods of expression, production, and purification of recombinant antibodies described above.

Specific examples of IL-6 variants are disclosed in Brakenhoff et al., J. Biol. Chem. (1994) 269, 86-93, Savino et al., EMBO J. (1994) 13, 1357-1367, W096-18648, and W096-17869.

The IL-6 partial peptide or IL-6 receptor partial peptide used in the present invention is a substance that has binding activity with the IL-6 receptor or IL-6, respectively, and does not transmit the biological activity of IL-6. That is, the IL-6 partial peptide or IL-6 receptor partial peptide binds to the IL-6 receptor or IL-6 and by capturing them, specifically inhibits IL-6 binding to the IL-6 receptor. As a result, the biological activity of IL-6 is not transmitted and thus signal transduction by IL-6 is blocked.

An IL-6 partial peptide or IL-6 receptor partial peptide is a peptide consisting of all or part of the amino acid sequence of the region involved in the binding of IL-6 with the IL-6 receptor in the amino acid sequence of IL-6 or the IL-6 receptor. Such peptide consists of usually 10 to 80, preferably 20 to 50, more preferably 20 to 40 amino acid residues.

An IL-6 partial peptide or IL-6 receptor partial peptide can be prepared by identifying the region in the amino acid sequence of IL-6 or the IL-6 receptor that is involved in the binding of IL-6 with the IL-6 receptor, and using a commonly known method, such as genetic engineering or peptide synthesis, based on all or part of the amino acid sequence of the identified region.

To prepare an IL-6 partial peptide or IL-6 receptor partial peptide by genetic engineering, the DNA sequence encoding the desired peptide can be incorporated into an expression vector and the peptide can be obtained in accordance with the methods of expression, production, and purification of recombinant antibodies described above.

To prepare an IL-6 partial peptide or IL-6 receptor partial peptide by peptide synthesis, a method usually used in peptide synthesis, such as solid-phase synthesis or liquid-phase synthesis, can be used.

Specifically, it can be prepared in accordance with the method described in Zoku Iyakuhin no Kaihatsu (Development of Drugs-Continued), Vol. 14, Peptide Synthesis, Haruaki Yajima (ed.), Hirokawa Shoten, 1991. As a solid-phase synthesis method, for example, a method of elongating a peptide chain by binding an amino acid corresponding to the C terminus of the peptide to be synthesized to a support that is insoluble in an organic solvent, and alternately repeating a reaction of condensing the amino acid protected with an appropriate protective group on the α-amino group and side chain functional group one amino acid at a time in the order from the C terminus to the N terminus and a reaction of eliminating the protective group on the α-amino group of the amino acid or peptide bound to the resin, is used. Solid-phase peptide synthesis methods are broadly classified into the Boc and Fmoc methods based on the type of protective group used.

After synthesizing the peptide of interest in this way, a deprotection reaction and a cleavage reaction of the peptide chain from the support are performed. For the cleavage reaction with the peptide chain, hydrogen fluoride or trifluoromethanesulfonic acid can be usually used in the Boc method, and TFA in the Fmoc method. In the Boc method, for example, the above protective peptide resin is treated in hydrogen fluoride in the presence of anisole. Then, the peptide is recovered by elimination of the protective group and cleavage from the support. This is lyophilized to obtain a crude peptide. On the other hand, in the Fmoc method, for example, the deprotection reaction and the cleavage reaction of the peptide chain from the support can be performed in TFA by the same operation as above.

The obtained crude peptide can be separated and purified by applying it to HPLC. The elution can be performed under optimal conditions using a water-acetonitrile solvent usually used for protein purification. The fraction corresponding to the peak of the chromatographic profile obtained is separated and lyophilized. The peptide fraction thus purified is identified by molecular weight analysis by mass spectrum analysis, amino acid composition analysis, amino acid sequence analysis, or the like.

Specific examples of IL-6 partial peptides and IL-6 receptor partial peptides are disclosed in Japanese Patent Laid-Open No. 2-188600, Japanese Patent Laid-Open No. 7-324097, Japanese Patent Laid-Open No. 8-311098 and U.S. Pat. No. 5,210,075.

The antibody used in the present invention may be a conjugated antibody bound to various molecules such as polyethylene glycol (PEG), radioactive substances, and toxins. Such conjugated antibody can be obtained by chemically modifying the obtained antibody. The methods for modifying antibodies have already been established in the art. The “antibody” in the present invention also includes these conjugated antibodies.

In the present invention, the IL-6 inhibitor is preferably an anti-IL-6 receptor antibody, and specific examples thereof include tocilizumab, sarilumab, and satralizumab.

The therapeutic or prophylactic agent for peripartum cardiomyopathy and the inhibitor of cardiac remodeling associated with peripartum cardiomyopathy of the present invention can be used in the treatment of cardiomyopathy. “Peripartum cardiomyopathy” means heart failure that develops during pregnancy and puerperium in women with no history of heart disease, and indicates a condition similar to dilated cardiomyopathy, but is distinguished as a different disease than dilated cardiomyopathy. In the WHO's definition and classification of cardiomyopathies, peripartum cardiomyopathy is classified as a secondary cardiomyopathy. In the American Heart Association (AHA)'s definition, peripartum cardiomyopathy (postpartum cardiomyopathy) is an acquired form of primary cardiomyopathy (which has a main lesion in the myocardium). In heart failure, as the disease stage progresses, dilatation of the left ventricle, decrease in contractility, and fibrosis of the myocardium occurs, and these changes are called “cardiac remodeling.” “Heart failure” refers to a condition in which palpitations, shortness of breath, fatigue, edema of the feet, and the like are gradually observed due to the heart not functioning adequately, and if left untreated, the patient eventually becomes unable to lie down due to difficulty breathing. The degree and speed of progression varies from person to person, but generally becomes more severe and refractory over time. Sudden onset and deterioration are not infrequent, and it is a dangerous condition which can eventually lead to death due to a lack of oxygen, or concurrent illnesses such as arrhythmia.

In the present invention, “treatment or prevention of peripartum cardiomyopathy” means treating or preventing reduced cardiac function caused by peripartum cardiomyopathy, as well as treating or preventing acute heart failure symptoms (such as dyspnea, cough, edema, general malaise, palpitations, shock, and disturbance of consciousness) and chronic heart failure symptoms (such as exertional breathlessness, edema, and palpitations) of partum cardiomyopathy.

In the present invention, “inhibition or improvement of cardiac remodeling” means stopping the progression of left ventricular dilatation, decrease in contractility, and myocardial fibrosis associated with the progression of the disease stage of heart failure, or improving these conditions compared to before drug administration.

The mineralocorticoid receptor (MR) is a receptor having equal affinity for mineralocorticoids (aldosterone) and glucocorticoids (cortisol), and is expressed in many tissues, such as the kidneys, colon, heart, central nervous system (hippocampus), brown adipose tissue and sweat glands. In the present invention, “IL-6 signaling pathway in the perinatal maternal heart via the neuronal mineralocorticoid receptor” means the pathway in which the cardiac plasma aldosterone concentration increases in the perinatal maternal heart, and IL-6 concentration in cardiac tissue increases due to the activation of central nervous system MR, which results in myocardial damage.

In the present specification, peripartum cardiomyopathy is diagnosed, for example, as heart failure symptoms that have newly appeared from late pregnancy to within 5 months postpartum in women with no history of heart disease and no other cause for developing heart failure. Examples of the symptoms include dilated cardiomyopathy-like conditions with reduced left ventricular ejection fraction (EF), such as less than 45%, and increased blood concentration of brain natriuretic peptide (BNP). Peripartum cardiomyopathy can be diagnosed based on, for example, BNP measurements, chest Xp/CT, echocardiography, and electrocardiography. In addition, peripartum cardiomyopathy can be diagnosed based on the results of cardiac CT, cardiac MRI, coronary angiography, or myocardial biopsy to rule out other cardiomyopathies.

Childbirth may be natural or may involve a cesarean section. Natural childbirth is divided into three main phases: in the first phase, labor pains occur, the cervix gradually opens and the fetus moves into the vagina, in the second phase, the fetus is delivered, and in the third phase, the placenta is delivered. “Postpartum” refers to the period when the third stage of childbirth is completed, and “after delivery” refers to “postpartum” or the period when the fetus is delivered by cesarean section. Examples of the subject of treatment in the present invention include women in late pregnancy (after 22 weeks of pregnancy) to within 5 months after delivery. In one aspect of the present invention, women in the lactation period after delivery are included as the subject. The “perinatal period” refers to the period from “22 weeks pregnancy to less than 7 days of age,” and the “puerperal period” is the period after delivery until the mother recovers, usually 6 to 8 weeks after delivery. The “lactation period” is the period from after delivery to weaning, when the infant is breastfed, and refers to approximately one year after birth. In one aspect of the present invention, lactating women are included as the subject, whether or not the lactating women are actually breastfeeding, including women who have stopped breastfeeding, for example, upon treatment. In addition, in one aspect of the present invention, women within 5 months postpartum are included as a subject of treatment in the present invention.

As described later, the present inventors have found that hypertrophic changes occur in the maternal heart postpartum, particularly during lactation. In one aspect of the present invention, the above lactating women can be targeted to suppress the onset or exacerbation of peripartum cardiomyopathy during lactation. In another aspect of the present invention, treatment of chronic symptoms of peripartum cardiomyopathy can also be continued beyond 5 months postpartum. The other aspect of the present invention can be suitably used particularly when breastfeeding is continued beyond 5 months postpartum.

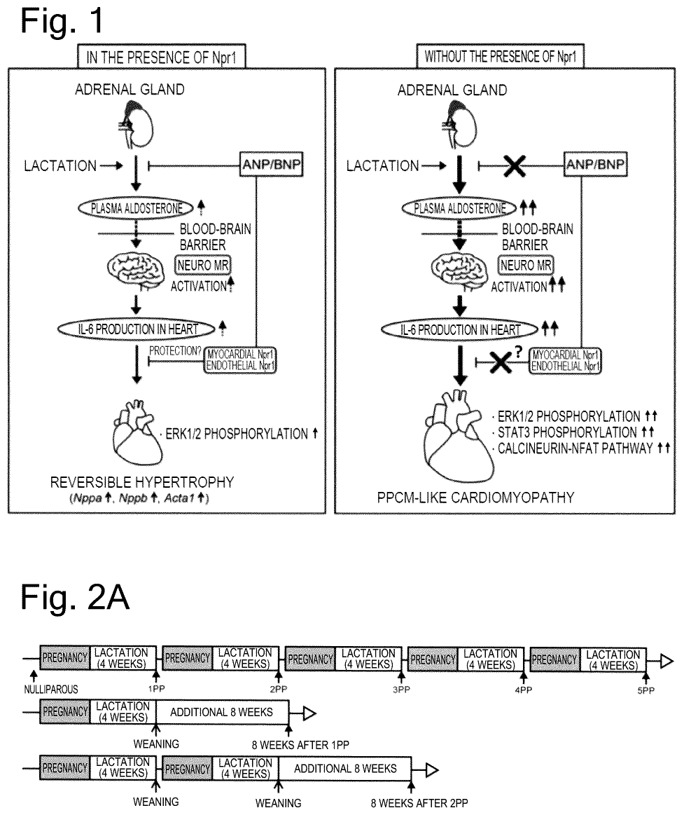

As described in the examples herein, NPR1, which is the receptor for ANP/BNP, is thought to be involved in the dynamic changes in the heart during pregnancy, childbirth, and postpartum, and Npr1^{-/-} mice can be used as a model animal for peripartum cardiomyopathy. The expected mechanism of action for cardiac hypertrophy in Npr1^{-/-} mice is shown in FIG. 1. When the ANP/BNP-NPR1 system is normal, the maternal heart develops reversible hypertrophy, accompanied by an increased phosphorylation of ERK1/2 (extracellular signal-regulated kinase) proteins. The mRNA expression of Nppa, Nppb, and Acta1 is significantly increased during lactation. These hypertrophic changes in the maternal heart are thought to be caused by increased plasma aldosterone levels and IL-6 production in the heart. However, deficiency of the ANP/BNP-NPR1 system results in a peripartum cardiomyopathy-like excessive cardiac hypertrophy, accompanied by fibrosis, marked cardiac dysfunction, and activation of the calcineurin-nuclear factor of activated T-cells (NFAT) pathway in the heart postpartum. These changes in the maternal heart are likely caused by a significant increase in plasma aldosterone and a marked activation of IL-6-dependent pathways in the heart. These results mean that the ANP/BNP-NPR1 system protects the maternal heart from lactation-induced cardiac remodeling. It should be noted that MR indicates mineralocorticoid receptor in FIG. 1.

In the present invention, treatment by administration of IL-6 inhibitor can be performed in combination with other treatments. Examples of other treatments include drug therapy (administration of ACE inhibitors, angiotensin receptor blockers, (3 blockers, diuretics, bromocriptine, etc.), and particularly in acute cases, ventilator management, intra-aortic balloon pumping (IABP), and cardiopulmonary support devices (PCPS, V-A. bypass, ECMO).

In the present invention, administration of IL-6 inhibitor can be used to treat subjects diagnosed with peripartum cardiomyopathy. In addition, in the present invention, administration of IL-6 inhibitor can be used to prevent worsening of heart failure conditions in subjects diagnosed with peripartum cardiomyopathy. In one aspect of the present invention, IL-6 inhibitor can be administered as a prophylactic treatment at the next pregnancy of women who have been diagnosed with peripartum cardiomyopathy at a previous pregnancy. Here, the subject for the prophylactic treatment includes, but is not limited to, women who have normalized cardiac function by the time of their next pregnancy.

The IL-6 signaling inhibitory activity of the IL-6 inhibitor used in the present invention can be evaluated by a method usually used. Specifically, ³H-thymidine uptake by IL-6-dependent cells can be measured by culturing IL-6-dependent human myeloma cell line (S6B45, KPMM2), human Lennert's T lymphoma cell line KT3, or IL-6-dependent cells MH60.BSF2, adding IL-6 to them, and allowing an IL-6 inhibitor to be present at the same time. In addition, 125 I-labeled IL-6 bound to IL-6 receptor-expressing cells is measured by culturing U266, which are IL-6 receptor-expressing cells, adding 125 I-labeled IL-6 and simultaneously adding an IL-6 inhibitor. The IL-6 inhibitory activity of the IL-6 inhibitor can be evaluated by having a negative control group without IL-6 inhibitor in addition to the group with IL-6 inhibitor in the above assay system, and comparing the results obtained for both groups.

The agents such as the pharmaceutical composition, therapeutic agent and prophylactic agent of the present invention can be administered in the form of a pharmaceutical preparation, which can be administered orally or parenterally systemically or locally. For example, intravenous injection such as drip infusion, intramuscular injection, intraperitoneal injection, subcutaneous injection, suppository, enema, oral enteric coated drug, and the like can be selected, and the appropriate method of administration can be selected according to the age and symptoms of the patient. Effective doses are selected in the range of 0.01 mg to 100 mg per kg body weight per dose, preferably in the range of 1 mg to 2 mg per kg body weight, preferably from 8 mg per kg body weight or 12 mg per kg body weight. Alternatively, a dose of 1 to 1000 mg, preferably 100 to 200 mg, preferably 120 mg, 150 mg, or 200 mg per patient can be selected. The preferred dosage and method of administration includes methods of administration by intravenous injection such as drip infusion, subcutaneous injection, and the like, where, for example, the amount of free antibody present in the blood is the effective dose in the case of anti-IL-6 receptor antibodies, with specific examples including 0.5 mg to 40 mg per kg body weight per month (4 weeks), preferably 1 mg to 20 mg in a single to several divided doses, for example, at a dosing schedule of b.i.w., q1w, q2w, q4w, and the like. The dosing schedule can also be adjusted by extending the dosing interval from b.i.w. or q1w to q2w, q3w, or q4w, while monitoring the condition after transplantation and the evolution of the blood test results.

Pharmaceutically acceptable carriers such as preservatives and stabilizers may be added to the agents such as the pharmaceutical composition, therapeutic agent and prophylactic agent of the present invention. A pharmaceutically acceptable carrier may be a material which itself has a therapeutic or prophylactic effect on the symptoms of peripartum cardiomyopathy and a suppressive effect on cardiac remodeling associated with peripartum cardiomyopathy, or a material which does not have this suppressive effect, and means a material that can be administered together with the above agents. It may also be a material that has no pharmacological effect but has a synergistic or additive stabilizing effect when used in combination with an IL-6 inhibitor.

Examples of pharmaceutically acceptable materials include sterile water, saline, stabilizers, excipients, buffers, preservatives, surfactants, chelating agents (such as EDTA), and binders.

In the present invention, examples of surfactants include nonionic surfactants, and typical examples thereof include those having an HLB of 6 to 18, such as sorbitan fatty acid esters such as sorbitan monocaprylate, sorbitan monolaurate, sorbitan monopalmitate, and sorbitan monooleate; glycerol fatty acid esters such as glycerol monocaprylate, glycerol monomyristate, and glycerol monostearate; polyglycerol fatty acid esters such as decaglyceryl monostearate, decaglyceryl distearate, and decaglyceryl monolinoleate; polyoxyethylene sorbitan fatty acid esters such as polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, and polyoxyethylene sorbitan tristearate; polyoxyethylene sorbitol fatty acid esters such as polyoxyethylene sorbitol tetrastearate and polyoxyethylene sorbitol tetraoleate; polyoxyethylene glycerol fatty acid esters such as polyoxyethylene glyceryl monostearate; polyethylene glycol fatty acid esters such as polyethylene glycol distearate; polyoxyethylene alkyl ethers such as polyoxyethylene lauryl ether; polyoxyethylene polyoxypropylene alkyl ethers such as polyoxyethylene polyoxypropylene glycol, polyoxyethylene polyoxypropylene propyl ether, and polyoxyethylene polyoxypropylene cetyl ether; polyoxyethylene alkyl phenyl ethers such as polyoxyethylene nonyl phenyl ether; polyoxyethylene hydrogenated castor oil such as polyoxyethylene castor oil and polyoxyethylene hydrogenated castor oil; polyoxyethylene beeswax derivatives such as polyoxyethylene sorbitol beeswax; polyoxyethylene lanolin derivatives such as polyoxyethylene lanolin; and polyoxyethylene fatty acid amides such as polyoxyethylene stearic acid amide.

Examples of surfactants also include anionic surfactants, with typical examples including alkyl sulfates with an alkyl group having 10 to 18 carbon atoms, such as sodium cetyl sulfate, sodium lauryl sulfate, and sodium oleyl sulfate; polyoxyethylene alkyl ether sulfates with an average number of moles of ethylene oxide added of 2 to 4 and an alkyl group having 10 to 18 carbon atoms, such as sodium polyoxyethylene lauryl sulfate; alkyl sulfosuccinates with an alkyl group having 8 to 18 carbon atoms, such as sodium lauryl sulfosuccinate; natural surfactants, such as lecithin, and glycerophospholipids; sphingophospholipids such as sphingomyelin; and sucrose fatty acid esters with fatty acids having 12 to 18 carbon atoms.

One or a combination of two or more of these surfactants can be added to the agent of the present invention. The preferred surfactants used in the formulation of the present invention are polyoxyethylene sorbitan fatty acid esters such as polysorbate 20, 40, 60 or 80, with polysorbate 20 and 80 being particularly preferred. Polyoxyethylene polyoxypropylene glycols typified by poloxamer (such as Pluronic F-68 (registered trademark)) are also preferred.

The amount of surfactant added varies depending on the type of surfactant used, but in the case of polysorbate 20 or polysorbate 80, it is generally 0.001 to 100 mg/mL, preferably 0.003 to 50 mg/mL, and more preferably 0.005 to 2 mg/mL.

In the present invention, examples of buffers include organic acids such as phosphoric acid, citric acid, acetic acid, malic acid, tartaric acid, succinic acid, lactic acid, potassium phosphate, gluconic acid, caprylic acid, deoxycholic acid, salicylic acid, triethanolamine, and fumaric acid, phosphate buffer (dibasic sodium phosphate hydrate, sodium dihydrogen phosphate dihydrate), citrate buffer, carbonate buffer, Tris buffer, histidine buffer (L-histidine, L-histidine hydrochloride hydrate), and imidazole buffer.

A solution formulation may also be prepared by dissolving in an aqueous buffer known in the field of solution formulations. The concentration of the buffer is generally 1 to 500 mM, preferably 5 to 100 mM, and more preferably 10 to 20 mM.

The agent of the present invention may also contain other low molecular weight polypeptides, proteins such as serum albumin, gelatin and immunoglobulins, amino acids, sugars and carbohydrates such as polysaccharides and monosaccharides, and sugar alcohols.

In the present invention, examples of amino acids include basic amino acids, such as arginine, lysine, histidine, and ornithine, and inorganic salts of these amino acids (preferably in the form of hydrochloride salts or phosphate salts, i.e., amino acid phosphates). If a free amino acid is used, the preferred pH value is adjusted by the addition of an appropriate physiologically acceptable buffering substance, such as an inorganic acid, particularly hydrochloric acid, phosphoric acid, sulfuric acid, acetic acid, formic acid or a salt thereof. In this case, the use of a phosphate is particularly advantageous in that a particularly stable lyophilized product can be obtained. It is particularly advantageous when the preparation is substantially free of organic acids, such as malic acid, tartaric acid, citric acid, succinic acid, or fumaric acid, or when no corresponding anion (such as malic acid ion, tartaric acid ion, citric acid ion, succinic acid ion, and fumaric acid ion) is present. The preferred amino acids are arginine, lysine, histidine, or ornithine. Furthermore, acidic amino acids, e.g., glutamic acid and aspartic acid, and their salt forms (preferably sodium salts), neutral amino acids, e.g., isoleucine, leucine, glycine, serine, threonine, valine, methionine, cysteine, or alanine, or aromatic amino acids, e.g., phenylalanine, tyrosine, tryptophan, or the derivative N-acetyltryptophan, can also be used.

In the present invention, examples of sugars and carbohydrates such as polysaccharides and monosaccharides include dextran, glucose, fructose, lactose, xylose, mannose, maltose, sucrose, trehalose and raffinose.

In the present invention, examples of sugar alcohols include mannitol, sorbitol, and inositol.

If the agent of the present invention is an aqueous solution for injection, it can be mixed with saline or an isotonic solution containing glucose or other adjuncts (for example, D-sorbitol, D-mannose, D-mannitol, and sodium chloride), and the aqueous solution may be used in combination with an appropriate solubilizing agent (such as an alcohol (ethanol, etc.), a polyalcohol (propylene glycol, PEG, etc.), or a nonionic surfactant (polysorbate 80, HCO-50, etc.). If desired, diluents, solubilizing agents, pH adjusters, soothing agents, sulfur-containing reducing agents, antioxidants, and the like may be further contained.

In the present invention, examples of sulfur-containing reducing agents include those having a sulfhydryl group, such as N-acetylcysteine, N-acetylhomocysteine, thioctic acid, thiodiglycol, thioethanolamine, thioglycerol, thiosorbitol, thioglycolic acid and salts thereof, sodium thiosulfate, glutathione, and thioalkanoic acids having 1 to 7 carbon atoms.

In the present invention, examples of antioxidants include erythorbic acid, dibutylhydroxytoluene, butylhydroxyanisole, α-tocopherol, tocopherol acetate, L-ascorbic acid and salts thereof, L-ascorbic acid palmitate, L-ascorbic acid stearate, sodium hydrogen sulfite, sodium sulfite, triamyl gallate, propyl gallate and chelating agents such as disodium ethylenediamine tetraacetate (EDTA), sodium pyrophosphate, and sodium metaphosphate.

If necessary, the agent can be encapsulated in microcapsules (e.g., hydroxymethylcellulose, gelatin, and poly[methyl methacrylate] microcapsules), or used in a colloidal drug delivery system (liposomes, albumin microspheres, microemulsion, nanoparticles, nanocapsules, etc.) (see e.g., “Remington's Pharmaceutical Science 16th edition,” Oslo Ed., 1980). Methods for preparing agents into sustained-release agents are also known in the art, and may be applied to the present invention (Langer et al., J. Biomed. Mater. Res. (1981) 15, 167-277; Langer, Chem. Tech. (1982) 12, 98-105; U.S. Pat. No. 3,773,719; EP Patent Publication No. EP58481; Sidman et al., Biopolymers 1983, 22: 547-556; and EP133988).

The pharmaceutically acceptable carriers to be used are selected from the above as appropriate or in combination depending on the dosage form, but are not limited thereto.

The subject for the treatment in the present invention includes, but is not particularly limited to, animals (e.g., humans, livestock animal species, wild animals).

In the present invention, “to administer” includes administering orally and parenterally. Examples of oral administration include administration in the form of an oral agent, and dosage forms such as granules, powders, tablets, capsules, solvents, emulsions, or suspensions can be selected as the oral agent.

Examples of parenteral administration include administration in the form of an injection, and examples of injections include subcutaneous injections, intramuscular injections, and intraperitoneal injections. In addition, the effect of the method of the present invention can be achieved by introducing the gene containing the oligonucleotide to be administered into the organism using a gene therapy technique. The agent of the present invention can also be administered locally to the area to be treated. For example, it is also possible to administer by local injection during surgery, by use of a catheter, or by targeted gene delivery of DNA encoding the peptide of the present invention. The agent of the present invention may be administered concurrently with prescriptions at the onset of cardiomyopathy, such as catheterization procedures (PTCA, PCI), thrombolytic therapy (PTCR), and coronary artery bypass grafting (CABG).

All references cited herein are incorporated herein by reference.

### EXAMPLES

Hereinafter, the present invention will be described in more detail on the basis of Example, but the present invention is not limited to these Examples.

### 1. Mice Preparation

All animal experiments were approved by the Ethics Committee for Animal Experiments of the National Cerebral and Cardiovascular Center and conducted in accordance with the guidelines of the Physiological Society of Japan. Npr1^{-/-} mice were prepared at the Howard Hughes Medical Institute (University of Texas Southwestern Medical Center, Dallas) (Nature. (1995) 378, 65-68. doi:10.1038/378065a0). All mice used in the present study were of C57BL/6 background. The mice were housed in a group at 25° C. under 12-h light/12-h dark cycle, with unrestricted access to food and water. Female mice (8 weeks old) were used in the experiment. Mating was performed using several females for one male, and the female mice that were found to be pregnant were housed individually in separate cages. After delivery, they were housed in the same cage as the pups until the end of the lactation period, and no adjustment was made to the number of pups.

### 2. Statistical Analysis

All data are indicated as the mean±standard error of the mean. The animals were randomly assigned to experimental groups. Kaplan-Meier analysis, followed by log-rank test, was used to compare survival between mice. Pairwise comparison was made using two-tailed unpaired Student's t-test. Differences between three or more groups were analyzed using one-way or two-way analysis of variance with Tukey-Kramer post-test. For comparisons between tissue-specific knockout mice, one-way analysis of variance followed by Dunnett post-test was applied. In microarray analysis, differential gene expression was determined using the t-test. Multiple testing was adjusted in accordance with the Benjamini-Hochberg method, and the false discovery rate was set at 0.05. For all comparisons, a P value of less than 0.05 indicates statistical significance.

### 3. Confirmation of PPCM-Like Cardiac Remodeling in Postpartum Npr1-Knockout Mice

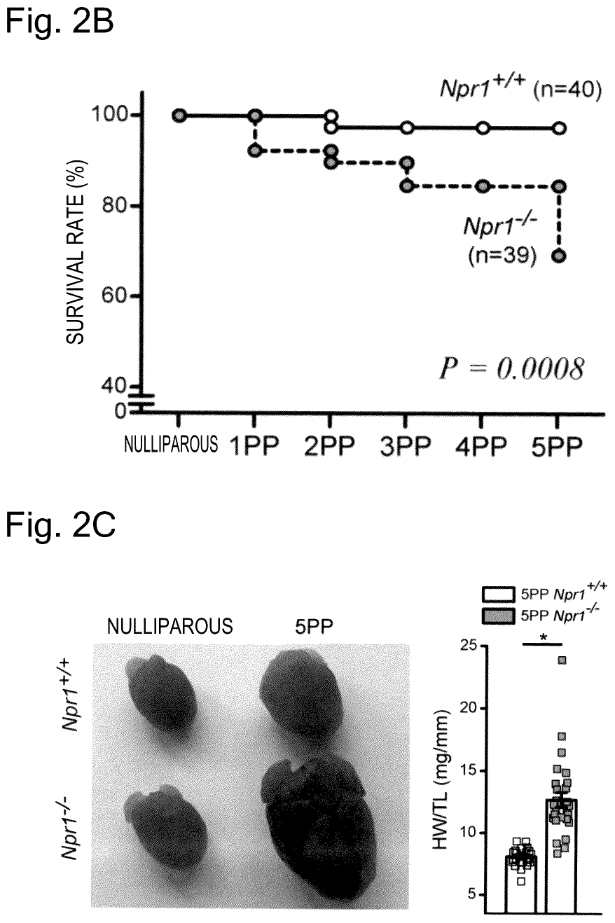

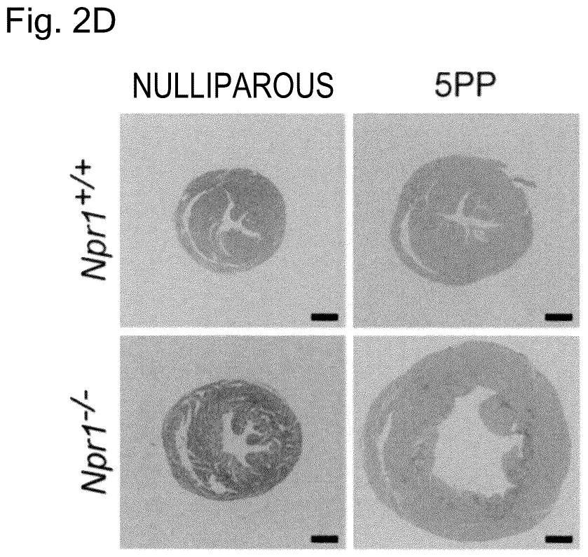

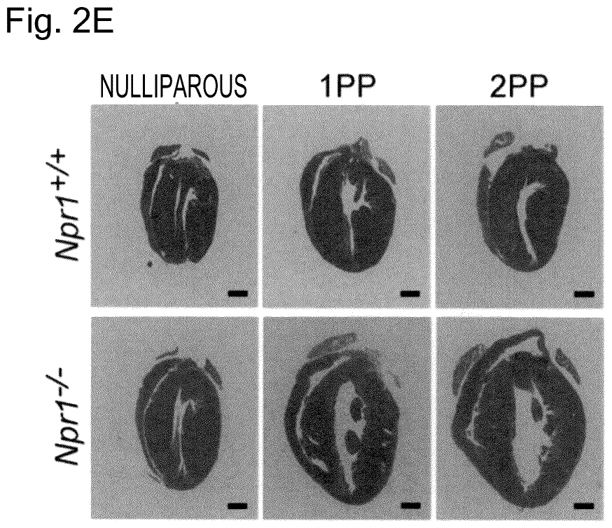

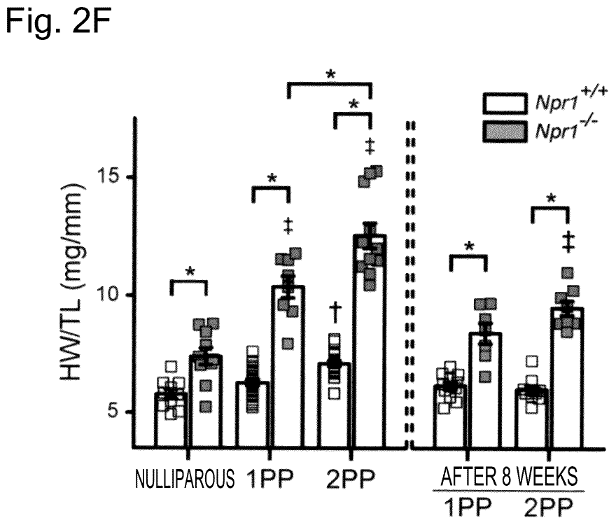

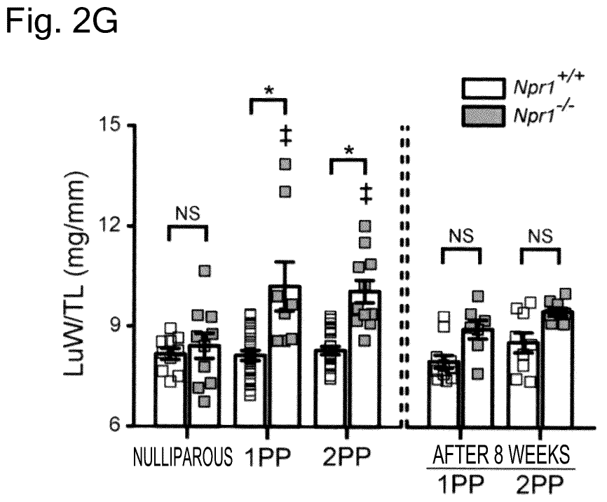

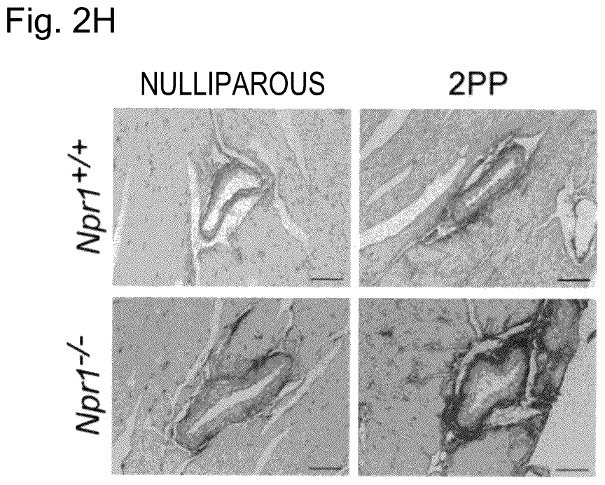

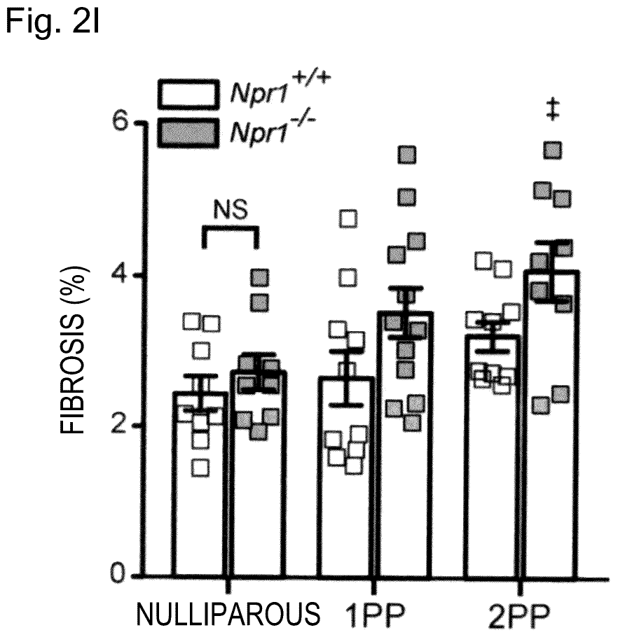

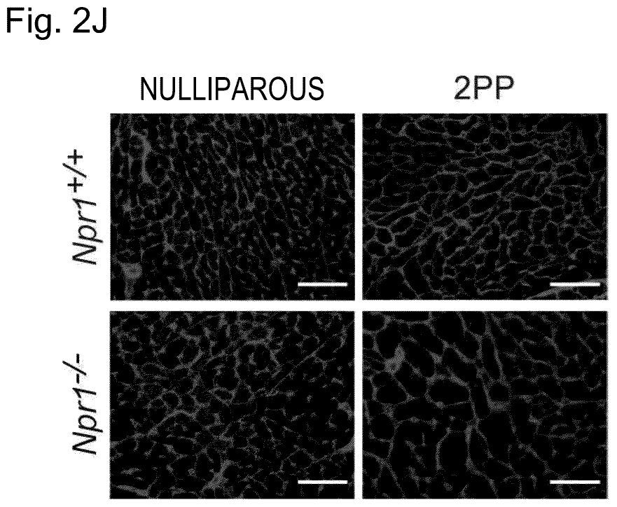

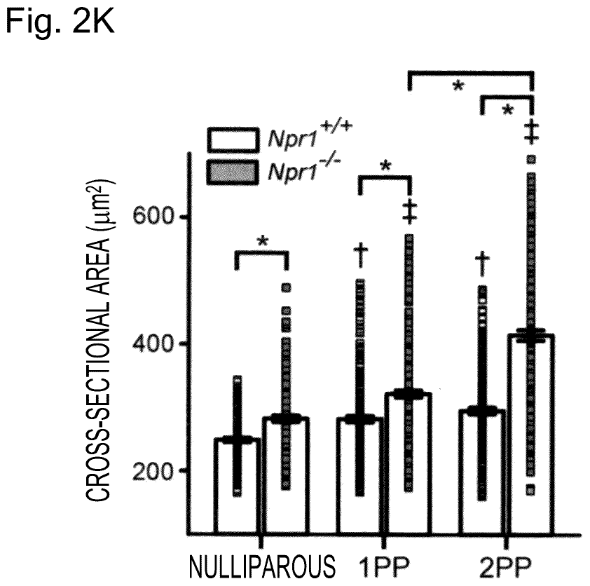

Npr1^{-/-} mice and wild-type mice (Npr1^{+/+} mice) were bred according to the experimental protocol shown in FIG. 2A. The survival rates after five pregnancy-lactation cycles are shown in FIG. 2B. The survival of Npr1^{-/-} mice was significantly lower than Npr1^{+/+} mice over consecutive pregnancy-lactation cycles (FIG. 2B; P=0.0008, vs. Npr1^{+/+} mice). Surprisingly, 75% of the Npr1^{-/-} mother animals that died during consecutive pregnancy-lactation cycles died during the lactation period. After the fifth consecutive pregnancy-lactation cycle, the hearts of the Npr1^{-/-} mice were markedly larger than the hearts of the Npr1^{+/+} mice, and were accompanied by increased lung weight, interstitial fibrosis, and increased mRNA expression of genes associated with cardiac hypertrophy (FIGS. 2C and 2D). The ratio of heart weight to tibial length (HW/TL) when nulliparous was slightly but significantly higher in Npr1^{-/-} mice than in Npr1^{+/+} mice (FIGS. 2E and 2F). However, the HW/TL after the first pregnancy-lactation cycle was significantly increased in postpartum Npr1^{-/-} mice (FIGS. 2E and 2F), and the HW/TL after the second consecutive pregnancy-lactation cycle (2PP) was significantly increased not only in postpartum Npr1^{-/-} mice, but in Npr1^{+/+} mice as well (FIGS. 2E and 2F). It should be noted that an increase in HW/TL means that cardiac hypertrophy has occurred.

In postpartum Npr1^{+/+} mice, the cardiac hypertrophy at 1PP and 2PP was fully restored to nulliparous levels by 8 weeks thereafter (FIG. 2F). In contrast, the cardiac hypertrophy in 2PP Npr1^{-/-} mice did not completely resolve by 8 weeks thereafter (FIG. 2F). The ratio of lung weight to tibial length (LuW/TL), which indicates lung congestion caused by heart failure, was similar between nulliparous Npr1^{+/+} mice and Npr1^{-/-} mice and remained virtually unchanged throughout the pregnancy-lactation cycles in Npr1^{+/+} mice but was significantly increased in 1PP and 2PP Npr1^{-/-} mice (FIG. 2G). Furthermore, a trend was confirmed that the recovery of LuW/TL up to 8 weeks postweaning was hampered in 2PP Npr1^{-/-} mice (P=0.064, vs. nulliparous Npr1^{-/-} mice; FIG. 2G). In postpartum Npr1^{+/+} mice, fibrotic areas in the heart were not increased (FIGS. 2H and 2I), but cardiomyocytes were significantly enlarged (FIGS. 2J and 2K). Both fibrotic area (FIGS. 2H and 2I) and cardiomyocyte size (FIGS. 2J and 2K) were significantly increased in Npr1^{-/-} mice at both 1PP and 2PP.

### 4. Confirmation of Induction of Cardiac Hypertrophy in Mice by Lactation

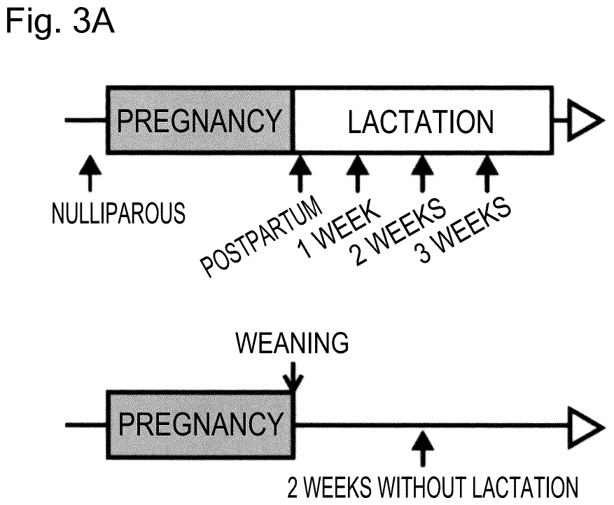

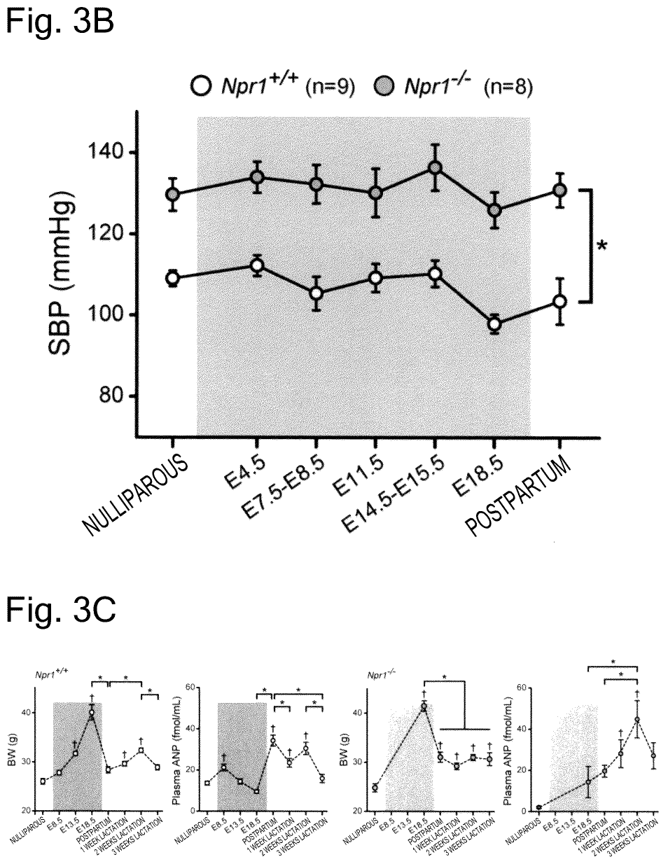

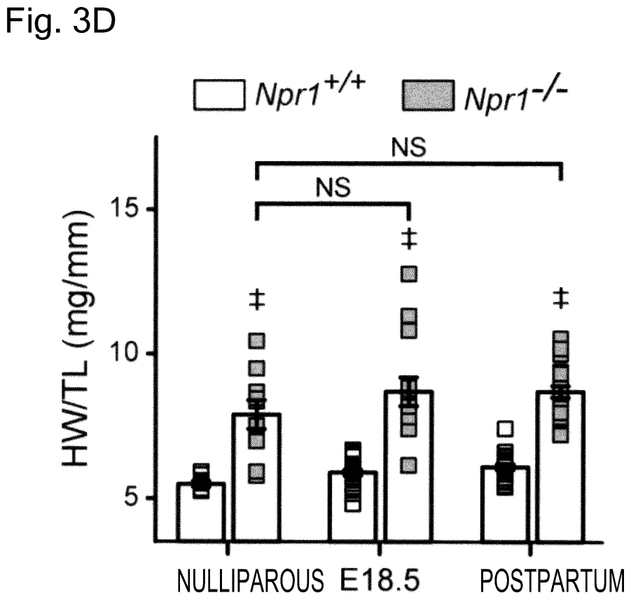

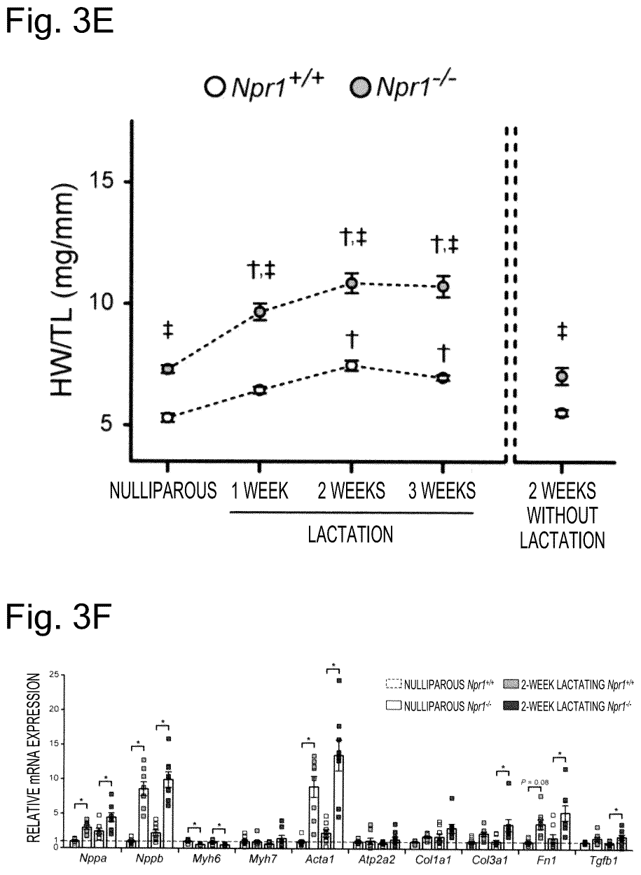

To determine which process (i.e., pregnancy, childbirth, or lactation) is responsible for cardiac hypertrophy in Npr1^{+/+} and Npr1^{-/-} mice, the maternal phenotype during all three processes was examined. The experimental protocol is shown in FIG. 3A. Twenty-two Npr1^{-/-} mice exhibited higher blood pressure in the nulliparous state than Npr1^{+/+} mice (FIG. 3B). Although mice lacking the proANP converting enzyme have been reported to develop pregnancy-induced hypertension (Chan, J. C., et al., Proc Natl Acad Sci USA. (2005) 102, 785-790. doi: 10.1073/pnas.0407234102), Npr1^{-/-} mice did not exhibit that phenotype (FIG. 3B). Maternal body weight was highest in late pregnancy for both Npr1^{+/+} mice and Npr1^{-/-} mice in the first pregnancy-lactation cycle (FIG. 3C). Plasma ANP peaked bimodally immediately after delivery and after 2 weeks lactation in Npr1^{+/+} mice, but peaked 2 weeks after delivery in Npr1^{-/-} mice (FIG. 3C). In contrast, HW/TL did not increase in late pregnancy or within 3 days after first delivery in either Npr1^{+/+} or Npr1^{-/-} mice (FIG. 3D). Furthermore, RCAN1 expression and ERK1/2 phosphorylation were not increased in late pregnancy (E18.5) in either Npr1^{+/+} or Npr1^{-/-} mice (data not shown). These findings mean that cardiac hypertrophy in perinatal mice is not induced by pregnancy-related fluid overload.

In contrast, HW/TL significantly increased within 2 weeks of lactation in primiparous Npr1^{+/+} and Npr1^{-/-} mice (FIG. 3E). mRNA expression levels of genes associated with cardiac hypertrophy (Nppa, Nppb, and Acta1) were significantly increased during lactation in both primiparous Npr1^{+/+} and Npr1^{-/-} mice (FIG. 3F). In comparison, mRNA expression of genes associated with fibrosis (Col3a1, Fn1, and Tgfb1) was significantly increased only in lactating Npr1^{-/-} mice (FIG. 3F). However, systolic blood pressure after 2 weeks lactation was not different from immediately after delivery in either Npr1^{+/+} or Npr1^{-/-} mice (data not shown).

The cardiac hypertrophy that occurred in Npr1^{+/+} or Npr1^{-/-} mice after the first pregnancy-lactation cycle was not observed when pups were removed immediately after birth and not breastfed (FIG. 3E; 2 weeks without lactation). Both the enlarged cardiomyocytes and the increased mRNA expression associated with cardiac hypertrophy were reduced in primiparous Npr1^{-/-} mice by removing pups and not breastfeeding (data not shown). However, preventing lactation did not affect cardiac function in either primiparous Npr1^{+/+} or Npr1^{-/-} mice (data not shown). These results indicate that lactation, but not pregnancy, induces cardiac hypertrophy in mice. That is, these results demonstrate the importance of the ANP/BNP-NPR1 system in potentially suppressing hypertrophic cardiac remodeling during lactation.

### 5. Effect of IL-6 Inhibitor on Lactation-Induced Cardiac Hypertrophy in Mice

Brain MR activation by aldosterone is known to regulate cardiovascular inflammation, oxidative stress, and sympathetic nerve activity. Therefore, a microarray analysis was then performed using RNA and cDNA derived from heart tissue from primipartum nulliparous and 2-week lactating Npr1^{+/+} and Npr1^{-/-} mice. In the hearts of Npr1^{+/+} and Npr1^{-/-} mice, expression level changes of 2-fold or more occurred in 3246 and 2336 probes, respectively. The results of the analysis are shown in the following table. The t-test was used for analysis, and P values were calculated by asymptotic; multiple comparison correction and Benjamini-Hochberg method. The adjusted P-value cutoff was set at 0.05 and the fold change cutoff was set at 2.0.

TABLE 1
Genes whose expression levels increase with lactation in Npr1^{+/+} mice (Top 20)

| | Gene | | Increase | |
|---|---|---|---|---|
| Probe Name | Symbol | Gene Name | (fold) | Adjusted P |
| A_55_P2006615 | Rbbp9 | Retinoblastoma Binding Protein 9 | 133.57 | 2.59E−05 |
| | | (Rbbp9) | | |
| A_51_P186547 | Pah | Phenylalanine Hydroxylase (Pah) | 101.69 | 2.82E−04 |
| A_55_P2038242 | Tchhl1 | Trichohyalin-Like 1 (Tchhl1) | 95.77 | 6.69E−06 |
| A_51_P325862 | Hrasls | HRAS-Like Suppressor (Hrasls) | 34.37 | 2.59E−05 |
| A_55_P1967659 | Lactb | Lactamase Beta (Lactb) | 33.83 | 1.38E−02 |
| A_66_P127463 | Gm5860 | Predicted Gene 5860 (Gm5860) | 27.79 | 4.79E−05 |
| A_51_P338998 | Prim2 | DNA Primase, p58 Subunit (Prim2) | 25.65 | 6.69E−06 |
| A_51_P163354 | Ogdhl | Oxoglutarate Dehydrogenase-Like | 25.30 | 1.03E−04 |
| | | (Ogdhl) | | |
| A_55_P2110738 | Pttg1 | Pituitary Tumor Transforming Gene 1 | 22.13 | 4.47E−05 |
| | | (Pttg1), Transcript Variant 2 | | |
| A_55_P1967231 | Stbd1 | Starch Binding Domain 1 (Stbd1) | 20.11 | 4.38E−03 |
| A_51_P159453 | Serpina3n | Serine (or Cysteine) Peptidase Inhibitor, | 20.09 | 1.23E−03 |
| | | Clade A, Member 3N (Serpina3n) | | |
| A_51_P146560 | Msln | Mesothelin (Msln) | 18.96 | 3.60E−04 |
| A_55_P1987499 | Pttg1 | Pituitary Tumor Transforming Gene | 15.83 | 7.59E−05 |
| | | 1(Pttg1), Transcript Variant 2 | | |
| A_51_P426195 | Nppb | Natriuretic Peptide B (Nppb), Transcript | 13.51 | 3.06E−04 |
| | | Variant 1 | | |
| A_55_P2056021 | Snap91 | Synaptosome Associated Protein 91 | 12.77 | 2.04E−04 |
| | | (Snap91), Transcript Variant 1 | | |
| A_55_P2078955 | Aqp8 | Aquaporin 8 (Aqp8), Transcript Variant | 12.77 | 3.24E−05 |
| | | 1 | | |
| A_55_P2067301 | Mgme1 | Mitochondrial Genome Maintenance | 11.90 | 7.82E−05 |
| | | Exonuclease 1 (Mgme1), Transcript | | |
| | | Variant 1 | | |
| A_52_P468068 | Tchh | Trichohyalin (Tchh) | 10.46 | 8.60E−05 |
| A_51_P279693 | Cyp1a1 | Cytochrome P450, Family 1, Subfamily | 9.36 | 1.69E−04 |
| | | a, | | |
| | | Polypeptide 1 (Cyp1a1), Transcript | | |
| | | Variant 1 | | |
| A_55_P2075313 | Zfp619 | Zinc Finger Protein 619 (Zfp619) | 9.31 | 3.90E−04 |

TABLE 2
Genes whose expression levels decrease with lactation in Npr1^{+/+} mice (Top 20)

| | Gene | | Decrease | |
|---|---|---|---|---|
| Probe Name | Symbol | Gene Name | (fold) | Adjusted P |
| A_55_P2105944 | Olfr224 | Olfactory Receptor 224 (Olfr224) | 28.23 | 1.16E−04 |
| A_55_P1966838 | Xaf1 | XIAP Associated Factor 1 (Xaf1), | 25.59 | 8.35E−03 |
| | | Transcript Variant 1 | | |
| A_55_P2094034 | Nrg2 | Neuregulin 2 (Nrg2) | 15.44 | 3.81E−05 |
| A_55_P2021810 | Arc | Activity Regulated Cytoskeleton | 10.63 | 6.43E−05 |
| | | Associated Protein (Arc), Transcript | | |
| | | Variant 1 | | |
| A_55_P2112986 | Klk1b22 | Kallikrein 1-Related Peptidase b22 | 10.33 | 8.93E−04 |
| | | (Klk1b22) | | |
| A_52_P354682 | Elovl7 | ELOVL Family Member 7, elongation | 10.25 | 7.01E−04 |
| | | of long chain fatty acids (Elovl7, | | |
| | | yeast) | | |
| A_66_P107583 | Rhox3a | Reproductive homeobox 3A (Rhox3a) | 9.89 | 3.33E−05 |
| A_51_P413147 | Klk1b3 | Kallikrein 1-Related Peptidase b3 | 9.84 | 4.09E−04 |
| | | (Klk1b3) | | |
| A_52_P565847 | AU018091 | Expressed Sequence AU018091 | 9.53 | 3.81E−05 |
| | | (AU018091) | | |
| A_55_P2186230 | Gm3317 | Predicted Gene 3317 (Gm3317) | 9.31 | 3.33E−05 |
| A_55_P2068615 | Bpifb4 | BPI Fold Containing Family B, | 9.14 | 4.57E−03 |
| | | Member 4 (Bpifb4) | | |
| A_55_P20044511 | Cd300lf | CD300 Antigen-Like Family Member | 8.99 | 3.01E−05 |
| | | F, | | |
| | | Transcript Variant (Cd300lf) | | |
| A_55_P2046877 | Foxq1 | Forkhead Box Q1 (Foxq1) | 8.66 | 9.08E−04 |
| A_55_P2161923 | Rabgap1 | RAB GTPase Activating Protein 1 | 8.07 | 3.33E−05 |
| | | (Rabgap1), Transcript Variant 2 | | |
| A_55_P1968028 | Tdgf1 | Teratocarcinoma-Derived Growth | 7.95 | 3.33E−05 |
| | | Factor 1 (Tdgf1) | | |
| A_55_P20044551 | Klra1 | Killer Cell Lectin-Like Receptor | 7.77 | 5.48E−05 |
| | | Subfamily A Member 1 (Klra1) | | |
| A_65_P10195 | Myl7 | Myosin, Light Chain Polypeptide 7, | 7.65 | 1.10E−03 |
| | | Regulatory (Myl7) | | |
| A_55_P2170350 | Klra22 | Killer Cell Lectin-Like Receptor | 7.33 | 3.00E−04 |
| | | Subfamily A Member 22 (Klra22) | | |
| A_55_P2003813 | Scn3b | Sodium Channel, Voltage-Gated Type | 7.30 | 5.77E−05 |
| | | III | | |
| | | Beta (Scn3b), Transcript Variant 2 | | |
| A_55_P2020203 | Sfrp5 | Secreted Frizzled Related Protein 5 | 7.18 | 3.75E−05 |
| | | (Sfrp5) | | |

TABLE 3
Genes whose expression levels increase with lactation in Npr1^{-/-} mice (Top 20)

| | Gene | | Decrease | |
|---|---|---|---|---|
| Probe Name | Symbol | Gene Name | (fold) | Adjusted P |
| A_55_P2078955 | Aqp8 | Aquaporin 8 (Aqp8), Transcript Variant | 15.15 | 1.03E−02 |
| | | 1 | | |
| A_51_P246854 | Acta1 | Actin, Alpha 1, Skeletal Muscle | 11.04 | 1.29E−02 |
| | | (Acta1), Transcript Variant 2 | | |
| A_52_P273120 | Myl1 | Myosin, Light Chain Polypeptide 1 | 9.49 | 4.38E−02 |
| | | (Myl1), Transcript Variant 3f | | |
| A_51_P314107 | Gsdma | Gasdermin A (Gsdma) | 7.97 | 1.33E−02 |
| A_55_P2065671 | Ccnb1 | Cyclin B1 (Ccnb1) | 7.23 | 1.73E−02 |
| A_51_P455897 | Pimreg | Family With Sequence Similarity 64, | 7.07 | 1.86E−02 |
| | | Member A (Fam64a) | | |
| A_51_P426195 | Nppb | Natriuretic Peptide B (Nppb), | 6.61 | 1.16E−02 |
| | | Transcript Variant 1 | | |
| A_55_P1956083 | Gpr68 | G Protein-Coupled Receptor 68 | 6.55 | 1.33E−02 |
| | | (Gpr68), Transcript Variant 1 | | |
| A_52_P232802 | Tbx15 | T-box 15 (Tbx15) | 6.49 | 1.33E−02 |
| A_55_P2050390 | Cdh22 | Cadherin 22 (Cdh22) | 6.28 | 1.33E−02 |
| A_55_P2048588 | Cdk1 | Cyclin Dependent Kinase 1 (Cdk1) | 6.16 | 1.92E−02 |
| A_51_P487999 | Sgol1 | Shugoshin-Like 1 (fission yeast) | 6.10 | 3.28E−02 |
| | | (Sgol1) | | |
| A_55_P1988083 | Prc1 | Protein Regulator Of Cytokinesis 1 | 6.04 | 1.84E−02 |
| | | (Prc1), Transcript Variant 1 | | |
| A_55_P2063736 | Lilr4b | Leukocyte Immunoglobulin-Like | 6.02 | 1.68E−02 |
| | | Receptor, Subfamily B Member 4B | | |
| | | (Lilr4b), Transcript Variant 2 | | |
| A_52_P162099 | Ckap2 | Cytoskeleton Associated Protein 2 | 5.91 | 1.50E−02 |
| | | (Ckap2) | | |
| A_55_P2132549 | Cd48 | CD48 Antigen (Cd48) | 5.90 | 2.08E−02 |
| A_55_P2005956 | Egfbp2 | Epidermal Growth Factor Binding | 5.87 | 1.53E−02 |
| | | Protein Type B (Egfbp2) | | |
| A_55_P2080530 | Poln | DNA Polymerase N of Transcript | 5.78 | 1.50E−02 |
| | | Variant 1 (Poln) | | |
| A_52_P99810 | Cx3cr1 | Chemokine (C-X3-C Motif) Receptor 1 | 5.77 | 1.70E−02 |
| | | (Cx3cr1) | | |
| A_52_P514061 | Padi4 | Peptidyl Arginine Deiminase Type IV | 5.75 | 2.15E−02 |
| | | (Padi4) | | |

TABLE 4
Genes whose expression levels decrease with lactation in Npr1^{-/-} mice (Top 20)

| | Gene | | Decrease | |
|---|---|---|---|---|
| Probe Name | Symbol | Gene Name | (fold) | Adjusted P |
| A_55_P2068615 | Bpifb4 | BPI Fold Containing Family B, | 27.27− | 1.14E−02 |
| | | Member 4 (Bpifb4) | | |
| A_55_P2009787 | Atp1a4 | ATPase, Na+/K+ Transporting, Alpha | 15.75 | 1.41E−02 |
| | | 4 Polypeptide (Atp1a4) | | |
| A_52_P413395 | Sln | Sarcolipin (Sln) | 12.70 | 3.02E−02 |
| A_55_P2121618 | Tox4 | TOX High Mobility Group Box | 8.97 | 1.92E−02 |
| | | Family Member 4 (Tox4) | | |
| A_55_P2073965 | BC049715 | cDNA Sequence BC049715 | 8.07 | 1.72E−02 |
| | | (BC049715) | | |
| A_55_P20044511 | Cd300lf | CD300 Antigen-Like Family Member | 7.38 | 1.33E−02 |
| | | F (Cd300lf), Transcript Variant 1 | | |
| A_55_P2020203 | Sfrp5 | Secreted Frizzled Related Protein 5 | 6.85 | 1.29E−02 |
| | | (Sfrp5) | | |
| A_52_P380379 | Ucp3 | Uncoupling Protein 3 (mitochondrial, | 6.83 | 1.33E−02 |
| | | proton carrier) (Ucp3) | | |
| A_55_P1959973 | Dcaf12l2 | DDB1 And CUL4 Associated Factor | 6.64 | 1.33E−02 |
| | | 12 Like 2 (Dcaf12l2) | | |
| A_55_P20044551 | Klra1 | Killer Cell Lectin-Like Receptor | 6.64 | 1.33E−02 |
| | | Subfamily A, Member 1 (Klra1) | | |
| A_55_P2064506 | Defb36 | Defensin beta 36 (Defb36) | 6.57 | 1.16E−02 |
| A_55_P2123854 | Zfhx2 | Zinc Finger Homeobox 2 (Zfhx2) | 6.48 | 1.34E−02 |
| A_55_P1968028 | Tdgf1 | Teratocarcinoma-Derived Growth | 6.38 | 1.33E−02 |
| | | Factor 1 (Tdgf1) | | |
| A_51_P326229 | Ddx25 | DEAD (Asp-Glu-Ala-Asp) Box | 6.37 | 1.33E−02 |
| | | Polypeptide 25 (Ddx25) | | |
| A_55_P2161923 | Rabgap1 | RAB GTPase Activating Protein 1 | 6.22 | 1.35E−02 |
| | | (Rabgap1), Transcript Variant 2 | | |
| A_55_P2180854 | Mrgprg | MAS- Related GPR, Member G | 6.19 | 1.33E−02 |
| | | (Mrgprg) | | |
| A_55_P2153620 | Ahnak | AHNAK Nucleoprotein (Desmoyokin) | 5.90 | 1.34E−02 |
| | | (Ahnak), Transcript Variant 3 | | |
| A_51_P431329 | Car3 | Carbonic Anhydrase 3 (Car3) | 5.89 | 1.29E−02 |
| A_55_P2003813 | Scn3b | Sodium Channel, Voltage-Gated Type | 5.89 | 1.33E−02 |
| | | III, Beta (Scn3b), Transcript Variant 2 | | |
| A_51_P338443 | Angptl4 | Angiopoietin-Like 4 (Angptl4) | 5.83 | 1.35E−02 |

Furthermore, significant (>1.5-fold) changes in gene expression occurred in 27 probes in the hearts of lactating Npr1^{-/-} mice compared to lactating Npr1^{+/+} mice (data not shown). Pathway analysis by Ingenuity Pathway Analysis (IPA) was performed to evaluate upstream regulators. The results of the evaluation on cytokines are shown in the table.

TABLE 5
Cytokines Predicted as Upstream Regulators in IPA Analysis

| | Acti- | | | |
|---|---|---|---|---|
| Upstream | vation | P Value of | Adjusted | Target Molecules in |
| Regulator | z-score^{a} | Overlap ^{b} | P ^{c} | Data Set |
| TNF | 1.958 | 2.00E−04 | 4.12E−03 | ADIPOQ, CFD, CIDEC, |
| | | | | CYP2E1, HP, IGF2, |
| | | | | PCK1, RETN |
| Ccl2 | | 1.25E−03 | 1.22E−02 | ADIPOQ, CFD |
| IL22 | | 5.97E−03 | 2.58E−02 | CYP2E1, HP |
| IL6 | | 6.15E−03 | 2.61E−02 | CFD, CYP2E1, HP, IGF2 |
| IL1B | −0.828 | 1.14E−02 | 3.57E−02 | ANXA9, CYP2E1, ENPP1, |
| | | | | HP |

^{a}A positive z-score indicates activation, and a negative z-score indicates inhibition.
^{b} P values of overlap are calculated using Fisher's exact test to evaluate overlap between genes in the data set and known targets of transcriptional regulators.
^{c} Adjusted by multiple comparison correction using the Benjamini-Hochberg method. Cutoff for adjusted P value: 0.05.

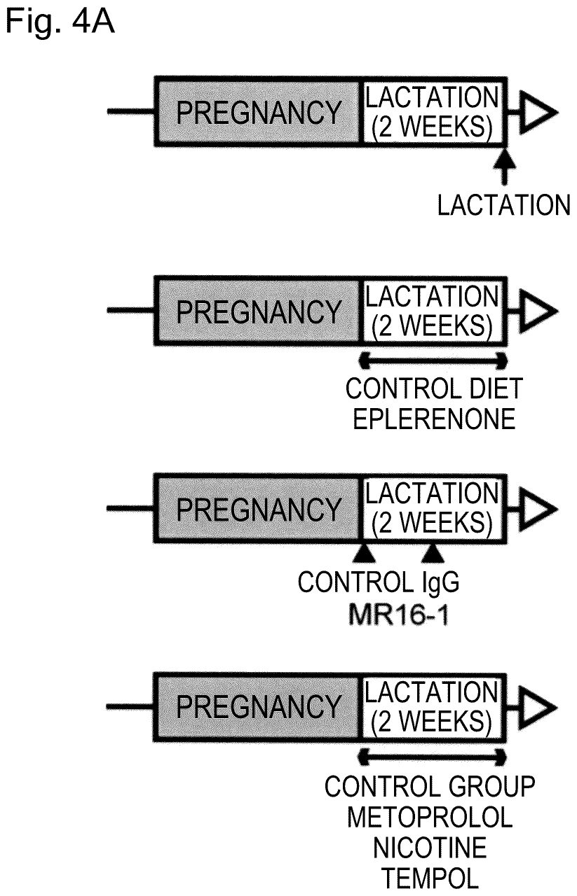

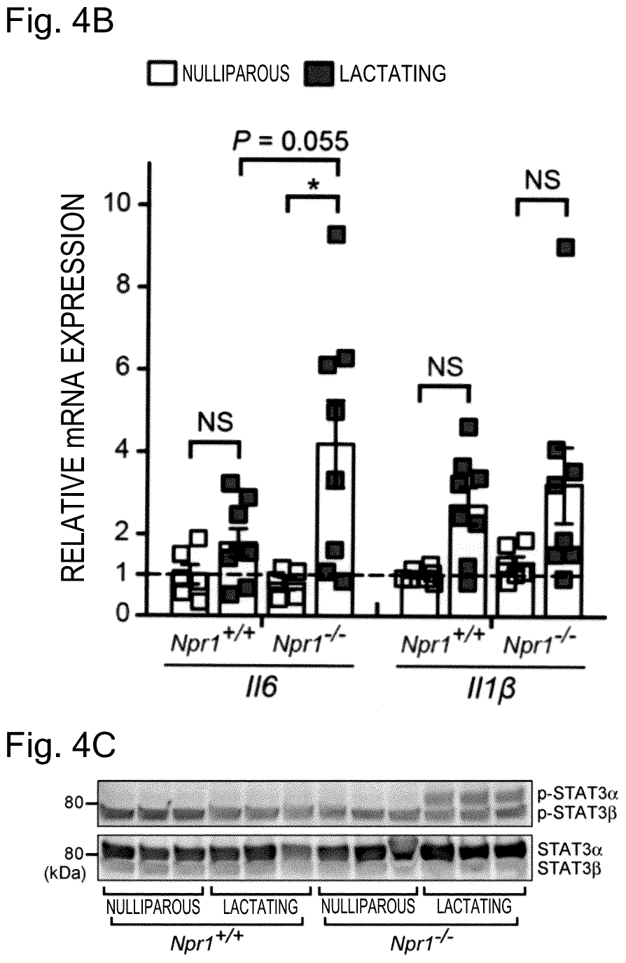

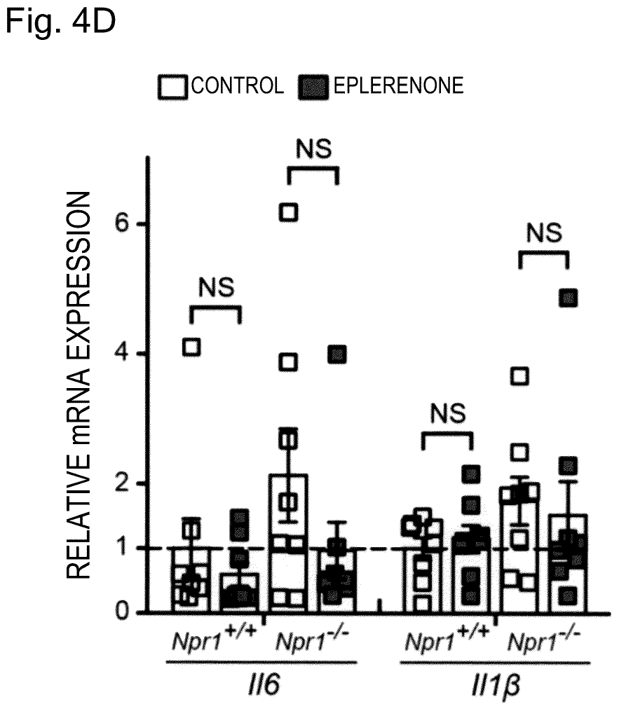

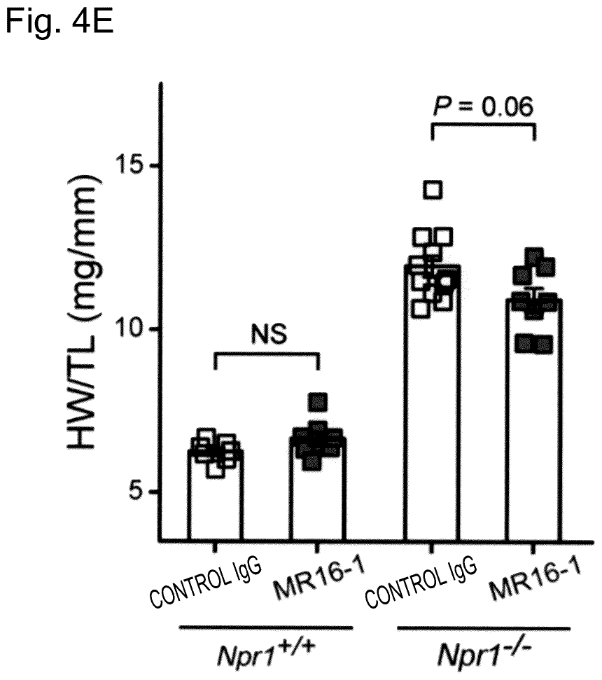

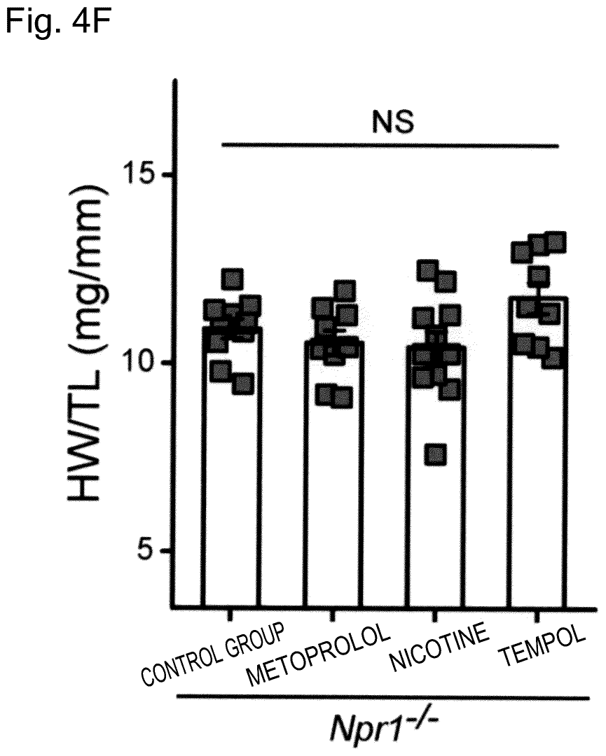

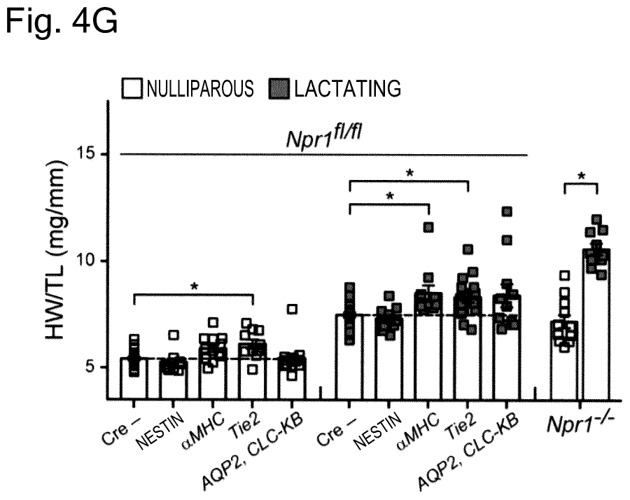

It was found that cardiac inflammatory cytokines may contribute to lactation-induced cardiac hypertrophy in Npr1^{-/-} mice. In addition, IL-6 and IL-1β have been reported to play an important role in the onset of cardiac hypertrophy (Non Patent Literature 10), and therefore the mRNA expression levels of IL-6 and IL-1β in the hearts of nulliparous and primiparous lactating Npr1^{+/+} and Npr1^{-/-} mice were examined. The experimental protocol is shown in FIG. 4A. Compared to nulliparous mice, cardiac 116 mRNA expression levels tended to increase in lactating Npr1^{+/+} mice, but was significantly increased in lactating Npr1^{-/-} mice (FIG. 4B). A tendency to increase during lactation was observed for MP mRNA levels in both primiparous Npr1^{+/+} and Npr1^{-/-} mice (FIG. 4B). In addition, the protein expression and degree of phosphorylation of signal-transducing transcription factor 3 (STAT3), a downstream target of IL-6, were studied by Western blotting. The primary and secondary antibodies used are listed in the table.

Table 6 Anti-STAT3 and Anti-Phospho-STAT3 Antibodies Used in Western Blotting

TABLE 6-1
Primary Antibodies

| | | Product | Dilution |
|---|---|---|---|
| | Manufacturer | Number | Ratio |
| Anti-Phospho-STAT3 | Cell Signaling Technology | 9145 | 1:1000 |
| Antibody | | | |
| Anti-STAT3 | Cell Signaling Technology | 8768 | 1:1000 |
| Antibody | | | |

TABLE 6-2
Secondary Antibodies

| | | Product | Dilution |
|---|---|---|---|
| | Manufacturer | Number | Ratio |
| Goat Anti-Rabbit IgG | Cell Signaling Technology | 7074 | 1:2000 |
| Antibody | | | |

Lactation markedly increased phosphorylated STAT3 (p-STAT3a) in the hearts of Npr1^{-/-} mice but not in Npr1^{+/+} mice (FIG. 4C). Lactation did not affect plasma concentration of IL-6 in either Npr1^{+/+} or Npr1^{-/-} mice, but the number of cardiac CD68 positive cells was higher in lactating Npr1^{-/-} mice than in lactating Npr1^{+/+} mice (data not shown). Eplerenone administration was found to reduce 116 expression in the hearts of primiparous Npr1^{-/-} mice (FIG. 4D).

Furthermore, to confirm the effect of anti-IL-6 receptor antibody (MR16-1) in Npr1^{+/+} and Npr1^{-/-} mice, a test was conducted by the following procedure. Npr1^{+/+} and Npr1^{-/-} mice were divided into control IgG administration groups (Npr1^{+/+} mice: n=7; Npr1^{-/-} mice: n=11) and MR16-1 administration groups (Npr1^{+/+} mice: n=8; Npr1^{-/-} mice: n=8). In all mice, control IgG or MR16-1 was administered intraperitoneally at 0.5 mg/mouse on the day of delivery (immediately after delivery) and one week after the start of lactation. The dose was standardized to 0.1 mL. Two weeks after the start of lactation, the heart weight and tibial length were measured to calculate the heart weight to tibia ratio (HW/TL). Weekly intraperitoneal injection of anti-IL-6 receptor antibody (MR16-1) showed a trend to suppress lactation-induced cardiac hypertrophy in primiparous Npr1^{-/-} mice, but not in Npr1^{+/+} mice (FIG. 4E).

A similar approach was used to study the administration of metoprolol 031-adrenoceptor antagonist), nicotine (a7-nicotinic acetylcholine receptor agonist) and tempol (radical scavenger) during the lactation period. Pharmacological modification of sympathetic or parasympathetic activity via any of the administrations did not suppress cardiac hypertrophy in Npr1^{-/-} mice (FIG. 4F). Furthermore, administration of tempol (radical scavenger) to lactating Npr1^{-/-} mice for 2 weeks did not suppress lactation-dependent cardiac hypertrophy (FIG. 4F).

### INDUSTRIAL APPLICABILITY

According to the present invention, a therapeutic or prophylactic agent for peripartum cardiomyopathy, particularly a therapeutic or prophylactic agent for peripartum cardiomyopathy postpartum or during lactation is provided.

1. A pharmaceutical composition for use in the treatment or prevention of peripartum cardiomyopathy, comprising an IL-6 inhibitor as an active ingredient.

2. The pharmaceutical composition according to claim 1, for use in a lactating subject.

3. The pharmaceutical composition according to claim 1 or 2, wherein the IL-6 inhibitor is an antibody that recognizes IL-6.

4. The pharmaceutical composition according to claim 1 or 2, wherein the IL-6 inhibitor is an antibody that recognizes an IL-6 receptor.

5. The pharmaceutical composition according to claim 3 or 4, wherein the antibody is a monoclonal antibody.

6. The pharmaceutical composition according to any one of claims 3 to 5, wherein the antibody is an antibody against human IL-6 or an antibody against a human IL-6 receptor.

7. The pharmaceutical composition according to any one of claims 3 to 6, wherein the antibody is a recombinant antibody.

8. The pharmaceutical composition according to any one of claims 3 to 7, wherein the antibody is a chimeric antibody, a humanized antibody, or a human antibody.

9. The pharmaceutical composition according to any one of claims 3 to 8, wherein the antibody is tocilizumab, satralizumab, or sarilumab.
